SEQUENCE LISTING (14 sequences)

SEQ ID NO: 1 - DNA, 23 bp, synthetic construct
  source 1..23: mol_type = other DNA
ggcatacacctactcaactacgg

SEQ ID NO: 2 - DNA, 22 bp, synthetic construct
  source 1..22: mol_type = other DNA
tgggcggtgtagaatcagagtc

SEQ ID NO: 3 - DNA, 22 bp, synthetic construct
  source 1..22: mol_type = other DNA
aatgcacagagtgtggcaaggc

SEQ ID NO: 4 - DNA, 22 bp, synthetic construct
  source 1..22: mol_type = other DNA
ctgctgatgtgcgaactgtagg

SEQ ID NO: 5 - DNA, 22 bp, synthetic construct
  source 1..22: mol_type = other DNA
tgccctcaagatgcacatccga

SEQ ID NO: 6 - DNA, 21 bp, synthetic construct
  source 1..21: mol_type = other DNA
gggacaggagaagggcttctc

SEQ ID NO: 7 - DNA, 22 bp, synthetic construct
  source 1..22: mol_type = other DNA
atctgcggcaaggcgttttcca

SEQ ID NO: 8 - DNA, 22 bp, synthetic construct
  source 1..22: mol_type = other DNA
gagccctcagatttgacctgtc

SEQ ID NO: 9 - DNA, 22 bp, synthetic construct
  source 1..22: mol_type = other DNA
gccaggtacatcgacttcctct

SEQ ID NO: 10 - DNA, 22 bp, synthetic construct
  source 1..22: mol_type = other DNA
tccatcctccagaccgagaagg

SEQ ID NO: 11 - DNA, 20 bp, synthetic construct
  source 1..20: mol_type = other DNA
ccgtgtgaaccatgtgactt

SEQ ID NO: 12 - DNA, 20 bp, synthetic construct
  source 1..20: mol_type = other DNA
ccaatccaaatgcggcatct

SEQ ID NO: 13 - DNA, 30 bp, synthetic construct
  source 1..30: mol_type = other DNA
ccagggctgcttttaactctggtaaagtgg

SEQ ID NO: 14 - DNA, 30 bp, synthetic construct
  source 1..30: mol_type = other DNA
atttccattgatgacaagcttcccgttctc

(19) United States
(12) Patent Application Publication
(10) Pub. No.: US 2026/0168986 A1
(43) Pub. Date: Jun. 18, 2026

(54) APPARATUS FOR OBSERVATION OF BIOLOGICAL ACTIVITY IN A SAMPLE AND A METHOD FOR QUANTIFYING CANCER INTRAVASATION EVENTS IN A BIOLOGICAL SAMPLE

(71) Applicant: Center for Neuromusculoskeletal Restorative Medicine Limited, Hong Kong (CN)

(72) Inventors: Anna Maria Blocki, Tai Po (CN); Rocky S. Tuan, Shatin (CN); Dai Fei Elmer Ker, Tai Po (CN); Christy Wing Tung Wong, Quarry Bay (CN)

(21) Appl. No.: 19/355,075

(22) Filed: Oct. 10, 2025

Related U.S. Application Data
(60) Provisional application No. 63/721,612, filed on Nov. 18, 2024.

(51) Int. Cl. G01N 33/50; C12M 1/00; C12M 1/12; C12M 1/34; C12M 3/06; C12N 5/073; C12N 5/09

(52) U.S. Cl. CPC G01N 33/5017; C12M 23/16; C12M 25/04; C12M 29/06; C12M 41/36; C12N 5/0605; C12N 5/0693; G01N 33/5064; C12N 2501/15; C12N 2533/54

(57) ABSTRACT

An apparatus for observation of biological activity in a sample and a method for quantifying cancer intravasation events in a biological sample includes a microfluidic device having a plurality of fluidic channel, including a center channel adjacent to a media channel defining a perfusable partition therebetween; wherein the center channel is arranged to facilitate mesenchymal transition (EMT) in biological cells housed within the center channel induced by a reagent supplied to the media channels.

### SEQUENCE LISTING

The sequence listing file entitled “sequence listing” having a size of 13,438 bytes and a creation date of Mar. 5, 2026, that was filed in the patent application is incorporated herein by reference in its entirety.

### TECHNICAL FIELD

This invention relates to an apparatus for observation of biological activity in a sample and a method for quantifying cancer intravasation events in a biological sample. Particularly, although not exclusively, the invention relates to a system and method for observing and quantifying an occurrence and characteristics of cancer intravasation events.

### BACKGROUND OF THE INVENTION

Lung cancer is the leading cause of cancer-related deaths worldwide. It is often only diagnosed at advanced metastatic stages when treatment options are limited, resulting in 5-year relative survival rates as low as only 9%. It is thus of high importance to develop physiologically relevant models of human metastatic lung cancer to enable a better study of the underlying pathological processes, identify suitable drug targets, and for high throughput drug screening. Microphysiological models of cancer metastasis have the potential to bridge the gap between 2D cell culture models, animal models and the actual clinical situation, thereby addressing the high attrition rates in drug development and to fulfill the transition toward personalized medicine.

Lung tumors can be divided into non-small cell lung cancer (NSCLC), comprising around 80-85% of all lung cancer cases, and small cell lung cancer (SCLC), comprising the other 15% of the cases. For epithelial lung cancer to metastasize, cancer cells from the primary solid tumor usually undergo five key stages, namely local invasion driven by a hallmark process called epithelial to mesenchymal transition (EMT), intravasation, dissemination, extravasation, and finally colonization as a secondary tumor. Among the five stages, EMT and intravasation are the rate limiting steps determining the number of circulating tumor cells with the potential to metastasize. Thus, tackling EMT, local invasion and intravasation could in principle minimize and/or prevent the systemic spread of cancer cells at its start and in this way improve the clinical outcome of lung cancer patients.

### SUMMARY OF THE INVENTION

In accordance with a first aspect of the present invention, there is provided an apparatus for observation of biological activity in a sample, comprising a microfluidic device having a plurality of fluidic channel, including a center channel adjacent to a media channel defining a perfusable partition there between; wherein a reagent supplied to the media channels induces epithelial-to-mesenchymal transition (EMT) in biological cells housed within the center channel.

In accordance with the first aspect, the center channel has a biological function of blood vessels.

In accordance with the first aspect, the center channel comprises a hollow luminal vasculature structure.

In accordance with the first aspect, the hollow luminal vasculature structure exhibits a permeability coefficient to a 40 kDa dextran of less than 1.0×10⁻⁶ cm/s

In accordance with the first aspect, the hollow luminal vasculature structure is formed by vasculogenesis.

In accordance with the first aspect, the hollow luminal vasculature structure is formed by culturing of human umbilical vein endothelial cells (HUVECs) to obtain a microvascular network of HUVECs.

In accordance with the first aspect, the human umbilical vein endothelial cells and the biological cells housed within the center channel are co-cultured.

In accordance with the first aspect, HUVECs and the biological cells are co-cultured in a hydrogel composite injected to the center channel.

In accordance with the first aspect, the reagent is an EMT-inducing cocktail composite.

In accordance with the first aspect, the EMT-inducing cocktail composite includes TGFβ1 and a macrophage condition medium.

In accordance with the first aspect, each of the plurality of fluidic channel includes an inlet port and an outlet port.

In accordance with the first aspect, the microfluidic device comprises a plurality of partitioning structures arranged to separate the center channel from the media channel.

In accordance with the first aspect, the partitioning structures includes triangular posts arranged in a regular interval.

In accordance with the first aspect, the microfluidic device includes a pair of media channels sandwiching the center channel.

In accordance with the first aspect, the pair of media channels are further connected with a common inlet port.

In accordance with the first aspect, the biological cells include cancer spheroids.

In accordance with the first aspect, the biological cells are selected from the group consisting of A549, NCI-H1975, and BEAS-2B cells

In accordance with the first aspect, the apparatus further comprises an imaging device adapted to capture images of the biological cells in the center channel to facilitate visualizing and recording intravasation events of the biological cells in the center channel.

In accordance with the first aspect, the imaging device includes a microscopic imager.

In accordance with the first aspect, the apparatus further comprising a processor arranged to analyze the captured images to identify cancer intravasation events in the biological cells based on extracted features from the images.

In accordance with the first aspect, the processor is a machine-learning processing engine.

In accordance with the first aspect, the machine-learning processing engine utilizes a random forest classifier, such as a fast random forest classifier, for image segmentation.

In accordance with a second aspect of the present invention, there is provided a method for quantifying cancer intravasation events in a biological sample, comprising the steps of: injecting and co-culturing HUVECs and cancer spheroids in the center channel of the microfluidic device in accordance with the first aspect; supplying the EMT-inducing cocktail composite in accordance with the first aspect in the media channel of the microfluidic device; capturing microscopic images of the center channel at a predetermined time interval; and processing the microscopic images using the machine-learning processing engine in accordance with the first aspect to quantify an occurrence and characteristics of cancer intravasation events.

In accordance with the second aspect, supplying the EMT-inducing cocktail composite reduces the expression of laminin and VE-cadherin in the microvascular network, thereby increasing permissiveness to cancer cell intravasation.

In accordance with the second aspect, the method is capable of distinguishing between biological cells with high metastatic potential and biological cells with low metastatic potential

The present invention provides a method for quantifying lung cancer intravasation events. The method involves culturing HUVECs and lung cancer spheroids in the center channel. HUVECs then form microvascular network (MVNs)—a 3D hollow luminal vascular network in the vicinity of cancer spheroids. After the formation of MVNs—macrophage conditioned medium cocktail is added to the media channels which induces the biological cells to undergo epithelial to mesenchymal transition (EMT) in the microfluidic device model. A machine learning model, trained on a dataset of lung cancer microvascular networks (MVNs) in microfluidic devices, is provided. The intravasation events of the EMT-induced cancer cells within the microfluidic device are analyzed using the machine learning model. The method further includes quantifying the number and characteristics of the lung cancer intravasation events based on the analysis results obtained by the machine learning model.

In addition, the present invention provides a 3D hollow luminal vasculature within the microfluidic device for quantifying lung cancer intravasation events. The device consists of a microfluidic chip with a culture chamber, i.e. the center channel, that includes hollow luminal vasculature structure that mimics blood vessels as well as lung cancer spheroids. The device also features a fluidic network connected to the culture chamber, enabling the introduction of a macrophage conditioned medium cocktail to induce EMT in the spheroids. Inlet and outlet ports are included for the controlled flow of media and fluids within the microfluidic device. The device incorporates imaging capabilities to visualize and record the intravasation events of lung cancer spheroids into the hollow luminal vasculature structure. Additionally, the device is integrated with a machine learning model for quantifying and analyzing the lung cancer intravasation events based on the captured images and features extracted from those images.

Furthermore, the present invention provides a machine learning model specifically designed for quantifying lung cancer intravasation events into a 3D hollow luminal vasculature metastatic microfluidic device. The model utilizes training data comprising a dataset of lung cancer intravasation events obtained from a microfluidic device. It incorporates feature extraction algorithms to extract relevant features from images or data representing the lung cancer intravasation events. The model utilizes a series of image processing steps that include but is not limited to image decompositing, thresholding, subtraction, compositing, and pixel-based classification using a random forest classifier for eventual segmentation of individual extravasated tumor cells and blood vessel structures followed by quantification of the occurrence and characteristics of lung cancer intravasation events. Validation and evaluation metrics are employed to assess the performance and accuracy of the machine learning model. The model is integrated with a 3D hollow luminal vasculature metastatic microfluidic device for real-time quantification and analysis of lung cancer intravasation events, based on the model's predictions and outputs.

The present invention provides advantages which highlight the combination and integration of macrophage-induced EMT, a 3D hollow luminal vasculature metastatic microfluidic device, and a machine learning model for quantifying and analyzing lung cancer intravasation events. By leveraging these techniques, the present invention provides a comprehensive solution for studying and understanding the process of lung cancer metastasis, particularly the intravasation events that occur during this process. This information is crucial for identifying predictable biomarkers, discovering new and highly specific drug targets, and facilitating drug screening processes to enable an earlier and more efficient treatment of cancer patients and thereby increase the likelihood of positive therapy outcomes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention will now be described, by way of example, with reference to the accompanying drawings in which:

FIG. 1 is a schematic diagram illustrating for observation of biological activity in a sample in accordance with an embodiment of the present invention.

FIG. 2 is a schematic diagram of the microfluidic device for observation of biological activity in a sample in accordance with an embodiment of the present invention.

FIG. 3 is a flow diagram illustrating a vasculogenesis process and co-culturing of biological cells in the center channel of the microfluidic device of FIG. 2.

FIG. 4A are images of protein arrays of EGM-2 (unconditioned culture medium) and Mφ-CM with chemiluminescently visualized signals for various secreted components. The levels of selected cytokines in Mφ-CM were quantified.

FIG. 4B illustrate quantified results of levels plotted as Log₂ fold-changes compared to control and classified according to their role either as EMT inducers or EMT suppressors, and either as pro- and/or anti-inflammatory cytokines.

FIG. 5A are representative micrographs depict cellular morphology. Images were analyzed for cellular circularity by ImageJ software, which was plotted after being normalized to the untreated control.

FIG. 5B are images of Live/Dead staining of treated A549 and quantification of cellular viability after 2 days of incubation with various EMT-inducers.

FIG. 5C are representative micrographs of A549 cells immunostained for epithelial marker E-cadherin, fluorescence intensity of signals for E-cadherin and vimentin was determined by measuring the Corrected Total Cell Fluorescence (CTCF), which was then normalized to respective controls.

FIG. 5D are representative micrographs of A549 cells immunostained for mesenchymal marker vimentin, fluorescence intensity of signals for vimentin was determined by measuring the Corrected Total Cell Fluorescence (CTCF), which was then normalized to respective controls.

FIG. 5E illustrates relative gene expression of SNAI1, SNAI2, TWIST1, ZEB1 and ZEB2 was determined by real-time qPCR. The relative mRNA levels of each gene (ΔΔCT values) were normalized to the respective levels of GAPDH (housekeeping gene).

FIG. 5F are representative micrographs of a 2D scratch migration assay. Dotted yellow lines indicate the initial gap area. Percentage of initial gap area free of cells after 12 hours of treatment was quantified by ImageJ software.

FIG. 5G illustrates 3D invasion assay of A549 spheroids embedded in collagen type I hydrogels after 24 hours of incubation. The degree of invasion of A549 cells into their surrounding environment was defined as the total area of invasion (indicated by yellow dotted line) divided by initial area of the spheroid (indicated by black circle) and normalized to control.

FIG. 5H illustrates quantification of cell nuclei using DAPI staining. A549 cells were incubated with TGF-β1, Mφ-CM or combination of both for two days. Section A shows representative images show DAPI-stained nuclei (blue). Nuclei were counted using particle analysis on ImageJ. Section B is a bar graph represents the average number of nuclei per field of view, normalized to controls.

FIG. 5I are representative micrographs depict the progression of A549 spheroid formation over a 3-day incubation period, showing formation of A549 spheroids in microwells over time.

FIG. 6A are photograph showing dimensions of the three-channel microfluidic device in accordance with an embodiment of the present disclosure.

FIG. 6B are representative micrographs showing the formation of MVNs by HUVECs in microfluidic devices with and without the introduction of EMT-IC on day 3 for 24 hours.

FIG. 6C illustrates quantification of total tubular length per field of view (FOV) and number of vascular junctions using ImageJ angiogenesis analyzer plug-in.

FIG. 6D are confocal microscopy Z-stack images of co-stained laminin and vinculin displayed in orthogonal view. Locations of cross-sections are indicated by white lines and orthogonal cross-sections are displayed on the sides.

FIG. 6E are confocal microscopy Z-stack images of co-stained VE-cadherin and F-actin displayed as z-stack images.

FIG. 6F illustrates western blot and densitometric band analysis of laminin normalized to their respective GAPDH levels of samples collected on day 4. Protein levels are displayed as fold-change and compared to control.

FIG. 6G illustrates western blot and densitometric band analysis of VE-cadherin normalized to their respective GAPDH levels of samples collected on day 4. Protein levels are displayed as fold-change and compared to control.

FIG. 6H is a schematic diagram of the cancer-on-a-chip model in accordance with an embodiment of the present disclosure.

FIG. 6I illustrates quantification of (A) Branch width, (B) tubular length and (C) number of junctions per field of view (FOV) using Image angiogenesis analyzer plug-in, and illustrates that EMT-IC (TGF-β1 & Mφ-CM) did not adversely affect stability and integrity of MVNs.

FIG. 7A are representative micrographs showing the formation of MVNs in the presence of A549 spheroids in microfluidic devices with and without the introduction of EMT-IC on day 3 for 24 hours. Initial tumor masses are outlined by yellow dashed lines.

FIG. 7B illustrates quantification of total tubular length per field of view (FOV) and number of vascular junctions using ImageJ angiogenesis analyzer plug-in.

FIG. 7C are confocal microscopy images of immunostained laminin, optionally overlayed with phase contrast (PhC) images. Borders of initial tumor masses are indicated by white dashed lines.

FIG. 7D illustrates western blot and densitometric band analysis of laminin normalized to their respective GAPDH levels of samples collected on day 4. Protein levels are displayed as fold-change as compared to control.

FIG. 7E are confocal microscopy images of co-stained VE-cadherin and F-actin. Borders of initial tumor masses are indicated by white dashed lines. Areas of close-ups are indicated by solid line rectangles, and close-ups are displayed in the row below. Close interaction between cancer cells and MVNs with clear borders is indicated by white arrowheads. Close interaction between cancer cells and MVNs with non-obvious borders is indicated by white asterisks.

FIG. 7F illustrates western blot and densitometric band analysis of VE-cadherin normalized to their respective GAPDH levels of samples collected on day 4. Protein levels are displayed as fold-change compared to control.

FIG. 7G are live cell images were taken every hour over a course of 24 hours starting on day 3 upon supplementation of EMT-IC or control medium into co-cultures of A549 spheroids and MVNs. Representative enlarged close-ups of timeframes of pre-labelled A549 cells (red) and MVNs (green) overlayed with their respective phase contrast images taken at selected timepoints are displayed. Migrating and invading cancer cells are indicated by dashed circles.

FIG. 7H illustrates live-cell fluorescence imaging of A549 cancer cell spheroids co-cultured with HUVEC-derived MVNs over 24 hours. Time-lapse live-cell imaging was performed hourly over a 24-hour period, starting on day 3 after supplementation with control medium into co-cultures of A549 spheroids and MVNs. Representative 10× magnification images display pre-labeled A549 cancer cells (red) and MVNs (green), along with corresponding phase contrast images. White arrows within boxed regions highlight migrating and invading cancer cells.

FIG. 7I illustrates live-cell fluorescence imaging of EMT-IC-facilitated A549 cancer cell migration and intravasation into MVNs over 24 hours. Time-lapse live-cell imaging was performed hourly over a 24-hour period, starting on day 3 after supplementation with EMT-IC into co-cultures of A549 spheroids and MVNs. Representative 10× magnification images display pre-labeled A549 cancer cells (red) and MVNs (green), along with corresponding phase contrast images. White arrows within boxed regions highlight migrating and invading cancer cells.

FIG. 7J illustrates live-cell fluorescence imaging of A549 cancer cell spheroids co-cultured with HUVEC-derived MVNs over 24 hours. Live cell images were taken every hour over a course of 24 hours starting on day 3 upon supplementation of control medium into co-cultures of A549 spheroids and MVNs. Representative enlarged close-ups of timeframes of pre-labelled A549 cells (red) and MVNs (green) overlayed with their respective phase contrast images taken every hour are displayed. Migrating and invading cancer cells are indicated by dashed circles.

FIG. 7K illustrates ILive-cell fluorescence imaging of A549 cancer cell spheroids co-cultured with HUVEC-derived MVNs over 24 hours. Live cell images were taken every hour over a course of 24 hours starting on day 3 upon supplementation of EMT-IC into co-cultures of A549 spheroids and MVNs. Representative enlarged close-ups of timeframes of pre-labelled A549 cells (red) and MVNs (green) overlayed with their respective phase contrast images taken every hour are displayed. Migrating and invading cancer cells are indicated by dashed circles.

FIG. 7L illustrates time-lapse live-cell imaging of A549 cancer cell migration and intravasation over 24 hours. Representative movies capture dynamic cell behaviors under different conditions over a 24-hour period. A and B are control condition, C and D are EMT-IC condition, showing A549 cell migration and intravasation into MVNs. White arrows highlight migrating and invading cancer cells.

FIG. 8A are 20× magnification confocal microscopy images of co-stained CD31 in control co-cultures and under EMT-IC exposure. Dashed white lines indicate initial A549 tumor masses. White rectangular frames indicate areas of close-ups, which are displayed in the rows below. Migratory non-intravasating A549 cancer cells are indicated by white arrow heads, while intravasating cancer cells are indicated by asterisks. To view intravasation events, MVNs were reconstructed and visualized using Qiber3D.

FIG. 8B are 20× magnification confocal microscopy images of co-stained F-actin in control co-cultures and under EMT-IC exposure, condition same as FIG. 8A.

FIG. 8C is a plot showing invasion area of A549 spheroids in microfluidic devices after 24 hours of incubation. The degree of invasion of A549 cells into their surrounding environment was defined as the total area of invasion divided by initial area of the spheroid and normalized to control.

FIG. 8D is a plot showing manual semi-quantification of intravasation events per spheroid.

FIG. 8E are high resolution confocal microscopy images (63×) including z-stack images displayed as orthogonal view and 3D projection of Control and EMT-IC conditions. Locations of cross-sections are indicated by white lines and orthogonal cross-sections are displayed on the sides.

FIG. 9A are 20× confocal microscopy images of control conditions in an experiment where EMT-IC (TGF-β1 with Mφ-CM) facilitated NCI-H1975 cancer cells intravasation into MVNs to various degrees. NCI-H1975 spheroids were co-cultured with MVNs in microfluidic devices and optionally supplemented with EMT-IC on day 3 for 24. On day 4 cells were stained for CD31 and F-actin.

FIG. 9B are 20× confocal microscopy images of EMT-IC-treated conditions in the same experiment of FIG. 10A. White dashed lines mark the original tumor boundaries and white boxes highlight regions shown in the close-up panels below. Migrating but non-intravasating cancer cells are marked with asterisks, while arrowheads indicate intravasating cells.

FIG. 9C illustrate invasion area of cancer spheroids in microfluidic devices after 24 hours of incubation in the same experiment of FIG. 10A. The degree of invasion of cancer cells into their surrounding environment was defined as the total area of invasion divided by initial area of the spheroid and was normalized to control.

FIG. 9D illustrate manual semi-quantification of intravasation events per spheroid in the same experiment of FIG. 10A.

FIG. 9E are high resolution confocal microscopy images (63×) including z-stack images displayed as orthogonal view of EMT-IC treated NCI-H1975 co-cultures with MVNs. Locations of cross-sections are indicated by white lines and orthogonal cross-sections are displayed on the sides.

FIG. 9F are 20× confocal microscopy images of control conditions in an experiment where EMT-IC (TGF-β1 with Mφ-CM) facilitated BEAS-2B cancer cells intravasation into MVNs to various degrees. BEAS-2B spheroids were co-cultured with MVNs in microfluidic devices and optionally supplemented with EMT-IC on day 3 for 24. On day 4 cells were stained for CD31 and F-actin.

FIG. 9G are 20× confocal microscopy images of EMT-IC-treated conditions in the same experiment of FIG. 10F. White dashed lines mark the original tumor boundaries and white boxes highlight regions shown in the close-up panels below. Migrating but non-intravasating cancer cells are marked with asterisks, while arrowheads indicate intravasating cells.

FIG. 9H illustrates invasion area of cancer spheroids in microfluidic devices after 24 hours of incubation in the same experiment of FIG. 10F. The degree of invasion of cancer cells into their surrounding environment was defined as the total area of invasion divided by initial area of the spheroid and was normalized to control.

FIG. 9I illustrates manual semi-quantification of intravasation events per spheroid in the same experiment of FIG. 10F.

FIG. 9J are high resolution confocal microscopy images (63×) including z-stack images displayed as orthogonal view of EMT-IC treated BEAS-2B co-cultures with MVNs. Locations of cross-sections are indicated by white lines and orthogonal cross-sections are displayed on the sides.

FIG. 10A illustrates a schematic overview of ML-assisted vessel segmentation (Blue coloured lines during training: background; magenta-coloured lines during training: vessels; blue solid color: automatic segmented vessel recognition).

FIG. 10B illustrates a schematic overview of ML-assisted tumor vessel contact quantification (Green solid colour: ML-segmented vessels; magenta colour: fluorescently labelled A549 cells).

FIG. 10C illustrates ML-assisted automatic identification and quantification of intravasation events per spheroid.

FIG. 10D illustrates segmentation settings for image training using Trainable Weka Segmentation. Overview of the parameters used for training images with Trainable Weka Segmentation, including selected training features, membrane thickness, and patch size. The Fast Random Forest classifier was utilized for segmentation. These settings were optimized to enhance accuracy and performance in distinguishing relevant structures.

FIG. 10E illustrates comparison of manual and ML-assisted quantification of intravasation events in A-Control and B-EMT-IC conditions. Heatmap on the right, C, showing intravasation events per spheroid for both methods, n=15 spheroids/condition.

FIG. 11A are confocal microscopy images of immunostained laminin, optionally overlayed with phase contrast (PhC) images. Borders of initial tumor masses are indicated by white dashed lines.

FIG. 11B illustrate western blot and densitometric band analysis of laminin normalized to their respective GAPDH levels of samples collected on day 4. Protein levels are displayed as fold-change as compared to control.

FIG. 11C are confocal microscopy images of co-stained VE-cadherin and F-actin. Borders of initial tumor masses are indicated by white dashed lines. Areas of close-ups are indicated by solid line rectangles, and close-ups are displayed in the row below. Close interaction between cancer cells and MVNs with clear borders is indicated by white asterisks. Close interaction between cancer cells and MVNs with non-obvious borders is indicated by white arrowheads.

FIG. 11D illustrate western blot and densitometric band analysis of VE-cadherin normalized to their respective GAPDH levels of samples collected on day 4. Protein levels are displayed as fold-change compared to control.

FIG. 11E are representative frames from live cell imaging showing the perfusion of MVN from one medium channel to the other on day 4.

FIG. 11F illustrates permeability coefficient of MVNs for FITC-PVP (40 kDa) perfused under control and EMT-IC conditions on day 4. Control: n=16, EMT-IC: n=17, collected from 3 biological replicates.

### DETAILED DISCLOSURE OF THE INVENTION

The inventors, through their experiments and trials, devised that in vivo animal models of cancer intravasation have low animal-to-human transitional rates from preclinical to clinical treatment, resulting in increasing concerns regarding the use of animals as predictive tools for human responses. These issues raised the need for physiologically relevant human in vitro models to investigate cancer biology and therapeutic development.

On the other hand, in vitro models such as Transwell migration/invasion assays, where a porous membrane seeded with a 2D layer of endothelial cells may be used to separate two chambers to observe trans-endothelial migration. Endothelial monolayers, however, are not able to recapitulate various functionalities, including full endothelial barrier properties, thus oversimplifying the 3D tumor microvasculature.

The inventors devised that for many years, cancer research were conducted in in vitro 2D cell culture and in vivo animal models. Although 2D monolayer cultures have been widely employed due to their high availability and reproducibility, these models are unable to model the etiology of the disease, to facilitate comprehensive cellular and environmental manipulation. Animal models also do not fully capture the complex biological processes that happen in the human body and have an inherently low resolution during real-time monitoring, limiting their read-out.

3D in vitro microphysiological models may provide a solution by bridging the gap between cell cultures and live tissues, enabling better control of the microenvironment while allowing the study of human physiology by using human cells. The inventors devised that various models of epithelial cancer intravasation may be used to more closely resemble the primary and metastatic TME in an in vivo-like manner, particular on-a-chip model. However, some example systems do not fully recapitulate the blood vessel anatomy as they focus on seeding endothelial on permeable membranes or hydrogel surfaces, thus still exhibiting properties of endothelial monolayers.

In addition, endothelial monolayers, however, lack various functional features of proper microvasculature, such as physiologically representative barrier functions. As barrier functions are one of the main obstacles cancer cells have to overcome to intravasate, the metastatic behavior of cancer cells cannot be properly modeled well with respect to the role of a functional microvasculature in these models.

One example cancer on-a-chip model utilized MVNs, which formed by vasculogenesis around cancer spheroids and exhibited improved barrier-properties. Nonetheless, this relied on the natural shedding of cancer cells from spheroids into MVNs and did not consider the process of EMT, thus rendering the study less physiologically relevant.

The inventors established approach to stabilize MVNs in microfluidic devices by utilization of macromolecular crowding (MMC) allowed the production of functional microvasculature-on-a-chip with hollow and circular lumina, apical-basal polarity and proper vascular barrier functions. Importantly, the measured permeability coefficients of MVNs in the lung cancer intravasation-on-a-chip was closely aligned with in vivo measurements in rat venular vessels (1.37±0.26×10⁻⁷ cm/s, indicating that EMT-IC did not compromise vascular barrier functions and that the MVNs maintain physiological permeability levels.

Although MVNs formed spontaneously by vasculogenesis, their density was determined by initial cell seeding density and was reproducible across devices and biological repeats. These MVNs were then utilized to establish the lung cancer cell intravasation-on-a-chip model and thus investigate cancer intravasation into functional microvasculature. This approach is straightforward, thereby ensuring ease of use across different laboratory settings with high verifiability and reproducibility. Furthermore, in contrast to a previously reported intravasation-on-a-chip model, the inventors considered EMT as a major intravasation driver and can be coupled with ML-assisted quantification. The inventors, therefore provide a novel model which is physiologically more representative and requires a shorter time to observe, visualize, and quantify intravasation events, and thus enabling more efficient and less time-consuming experimentation and screening.

The inventors devised that cancer-on-a-chip models may be powerful tools to investigate the tumor microenvironment and its contribution to cancer intravasation and metastasis. Preferably, they provide a controlled environment to simulate blood vessel-like conditions and enable the utilization of live-cell imaging to visualize intravasation events into artificial microvessels.

Although some example in vitro models have allowed the study of how various chemical and physical aspects influence the metastatic cascade, these models may consist of microchannels lined with a monolayer of endothelial cells, which does not fully recapitulate the blood vessel anatomy and lacks crucial features of functional microvessels, such as good physiologically representative vascular barrier functions.

Hence, it is preferable to provide a physiologically relevant model that recapitulates both events of local invasion from the primary tumor via EMT, followed by intravasation into the MVNs. In accordance with an embodiment of the present invention, there is provided a human microphysiological in vitro model of EMT-driven lung cancer intravasation in a microfluidic device coupled with machine learning (ML)-assisted quantification of intravasation events within. In this example, a robust EMT induction cocktail that was then applied to a co-culture system of lung cancer spheroids (microtumor masses), derived from A549 cells, embedded within 3D physiologically representative MVNs in microfluidic devices. In addition, live cell imaging, high-resolution microscopy, and ML-assisted vessel segmentation combined with co-localization analysis to detect intravasation events were utilized to visualize and quantify intravasation events, respectively. Advantageously, the present invention opens avenues to study physiologically relevant lung cancer-related pathological processes in high spatiotemporal resolution and to utilize the established platform technology for drug development and high throughput screening.

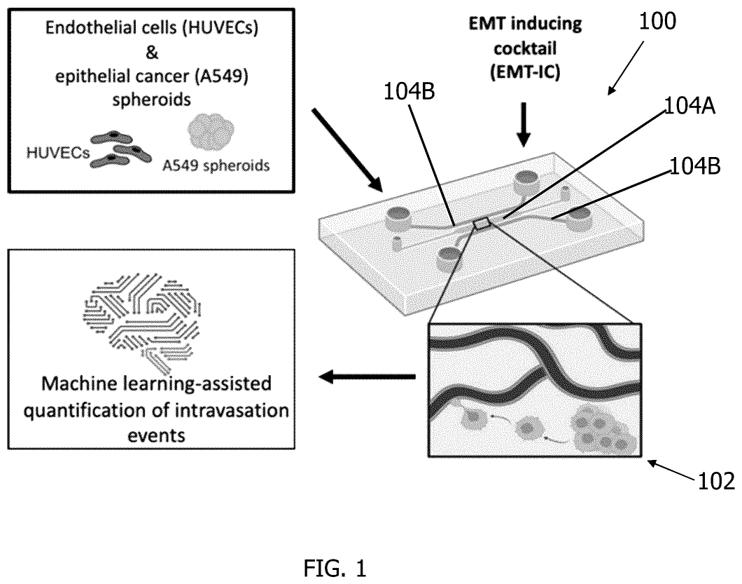

In accordance with FIG. 1 there is shown an example embodiment of an apparatus 100 for observation of biological activity 102 in a sample, comprising a microfluidic device 104 having a plurality of fluidic channels, including a center channel 104A adjacent to a media channel 104B defining a perfusable partition 104C therebetween; wherein the center channel 104A is arranged to facilitate mesenchymal transition (EMT) in biological cells housed within the center channel 104A induced by a reagent supplied to the media channels 104B.

In this example, the microfluidic device 104 may be use for observing EMT that may occur in the center channel 104A of the microfluidic device 104, in which the center channel 104A may house biological cells such as spheroids that undergo EMT, and EMT would occur in the center channel 104A and in vicinity of blood vessel-like structures.

For example, biological cells such as cancer spheroids may be injected into the center channel 104A through a corresponding inlet of the same channel, and the cancer spheroids remains within the center channel 104B during an experiment. Co-cultured in the center channel may be endothelial cells forming a microvascular network, which may mimic properties and function of a blood vessel. To initiate an EMT mechanism in the cancel spheroids, EMT inducing cocktail (IC) may be injected to one or both media channels 104B to trigger EMT in cancers cells within the center channel 104A which is spatially separate from the adjacent media channels 104B but is being perfusable.

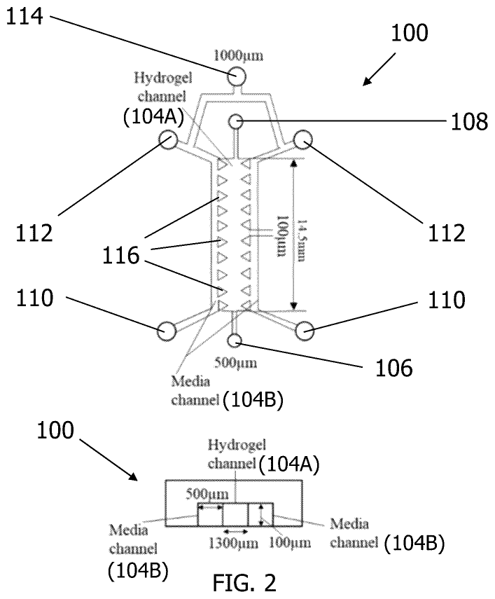

With reference also to FIG. 2, there is shown an example structure of the microfluidic device 100. In this example, the microfluidic device 100 is a 3-channel microfluidic chip with a center channel 104A and a pair of media channels 104B sandwiching the center channel 104A. The center channel 104A is connected with a cell inlet 106 and a cell outlet 108, and each of the media channels 104B is connected to a media inlet 110 and a media outlet 112. Optionally, the pair of media channels 104B are further connected with a common inlet port 114, therefore there are altogether 3 media inlets and 2 media outlets.

In addition, the microfluidic device 100 comprises a plurality of partitioning structures 116 arranged to separate the center channel 104A from the media channel(s) 104B. The fluidic channels 104A, 104B have a height of 100 μm and length of 14.5 mm with triangular posts 116 separating the center hydrogel channel 104A from the adjacent media channels 104B. The posts 116 are arranged in a regular interval, e.g. 100 μm apart. The posts 116 are provided to make sure that hydrogel injected to the center channel 104A are properly retained in the cavity sandwiched by these post structures 116.

The width of the center hydrogel channel 104A and media channels 104B is 1300 μm and 500 μm, respectively. Media inlet ports 110 and the cell inlet port 106 have a diameter of 1000 μm and 500 μm, respectively. As appreciated by a skilled person in the art, the dimensions of different features in the microfluidic device as abovementioned are exemplary only, and therefore may be varied in alternative embodiments for use in other applications.

In one example embodiment, the microfluidic device 100 may be fabricated by replica molding on a silicon wafer and soft lithography using PDMS (polydimethylsiloxane). In an experiment carried out by the inventor, a 100 μm layer of SU-8 3050 negative photoresist (Kayaku Advanced Materials, Massachusetts, USA) primer was spin-coated on a silicon wafer before being exposed to a photomask exhibiting the negative pattern of the channel structures designed by computer aided designs (CAD) for photolithography. The SU8 was then exposed to UV light (set as 20 mW/cm² at 365 nm) for 45 seconds, followed by the pattern developing. PDMS and curing agent (Sylgard 184, Dow Corning, Michigan, USA) were mixed at 10:1 (W/W) ratio and cast onto the SU8 master. After thermal curation at 60° C. for two hours, a positive replica-molded pattern on PDMS was separated from the wafer. Patterned PDMS was cut into individual devices and inlet and outlet ports were punched using 1 and 3 mm biopsy punchers. Next, the devices and glass slides were cleaned with 100% ethanol, water, and dried with a nitrogen gas air gun before being treated with oxygen plasma (Harrick Plasma, New York, USA) for 45 seconds to create covalent bonding between the glass slide and the PDMS device. Immediately after the PDMS pieces and glass slides were assembled, the channels were coated with 1 mg/ml of poly-L-lysine (PLL) (Molecular weight: 30,000-70,000) (Meryer, Shanghai, China, Cat #. 25988-63-0) dissolved in water for at least 20 minutes before autoclaving. PLL coating increased the hydrophilicity of the device enabling easy loading of the hydrogel.

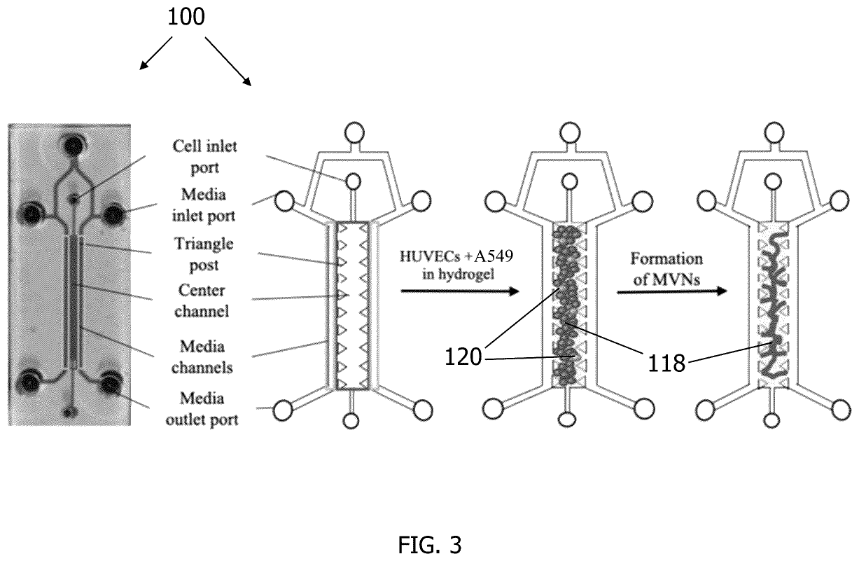

The PDMS microfluidic device 100 may be used for further forming the hollow luminal vasculature structure in the center channel 104A, e.g. by vasculogenesis. Preferably, the hollow luminal vasculature structure may be formed by culturing human umbilical vein endothelial cells (HUVECs) to obtain a microvascular network 118 of HUVECs in the center channel 104A, and more preferably, the human umbilical vein endothelial cells and the biological cells 120, i.e. cancer spheroids, housed within the center channel 104A are co-cultured, with reference also to the flow diagram of FIG. 3.

In one example embodiment, the microvascular network of HUVECs and cancer spheroids may be co-cultured in the center channel of the microfluidic device, in which both HUVECs and cancer spheroids are injected in the center channel. In an experiment carried out by the inventor, a solution of fibrinogen (Sigma-Aldrich, Cat #. F8630-5G) was prepared dissolved freshly in PBS at 15 mg/ml for each experiment. Thrombin solution (Sigma-Aldrich, Cat #. T4648) was prepared in 1% (w/v) BSA (Sigma-Aldrich, Cat #. A7906) in PBS solution at 100 U/ml and stored in aliquots at −20° C. HUVECs were resuspended in EGM-2 containing 6 U/ml of thrombin, the cell solution was mixed with fibrinogen solution at 1:1 ratio to achieve final concentrations at 7.5 mg/ml for fibrinogen, 3 U/ml for thrombin and 6×10⁶ cells/ml HUVECs. 300-cell A549 spheroids were formed as described under “spheroid formation” later. The spheroids were collected and resuspended in 200 μl of EGM-2 and 2 μl/droplets were pipetted onto a Petri dish for visualization and selection. Droplets with more than 10 spheroids were selected to be introduced quickly into the center channel with the mixture of HUVECs, fibrinogen solution as well as thrombin, and the device was placed at 37° C. in a humidified incubator for 1 hour to allow the fibrinogen to be polymerized by thrombin into a fibrin hydrogel. The hydrogel containing the HUVECs and cancer spheroids remained in the center channel during the culturing process.

Next, EGM-2 was added into the medium channels. Medium was changed on a daily basis. To introduce macromolecular crowding (MMC) into 3D cell culture, media containing Ficoll macromolecules (25 mg/ml Ficoll 400-Cytiva, Marlborough, MA, USA, Cat #. 17-0300-50; and 37.5 mg/ml of Ficoll 70-Cytiva, Cat #. 17-0210-10), were introduced from day 1 onwards. The composition of the Ficoll cocktail exhibiting a calculated fractional volume occupancy (FVO) of 17% and was demonstrated to enhance ECM deposition and basement formation around MVNs. On day 3, the co-culture was incubated with or without EMT-inducing cocktail also supplemented with 25 mg/ml of Ficoll 400 and 37.5 mg/ml of Ficoll 70 for 24 hours.

Other information related to materials and methods involved in the example experiment carried out by the inventors are further disclosed as follows:
Regarding general cell culture, human monocytic cell line THP-1 (ATCC TIB-202, Manassas, VA, USA) was maintained in Roswell Park Memorial Institute (RPMI) 1640 Medium, GlutaMAX™ Supplement (GIBCO, Life Technologies, Grand Island, NY, USA, Cat. #61870036), while A549 cells (ATCC, Cat. #CCL-185) were cultured in Dulbecco's Modified Eagle's Medium (DMEM) with 1 g/L glucose and GlutaMAX™ (GIBCO, Cat. #10567-014). Both culture media were supplemented with 1% of 100 U/ml penicillin and 100 μg/ml streptomycin (P/S) (GIBCO, Cat. #15140-122), and 10% fetal bovine serum (FBS) (GIBCO, Life Technologies, Cat. #16000044). Primary human umbilical vein endothelial cells (HUVECs, pooled) (ATCC, Cat #. PCS-100-013) and GFP-expressing HUVECs (TTFLUOR HUVECs) (Innport, Primera Planta, Spain Cat #. P20201) were cultured in Endothelial Growth Medium (EGM-2) (Lonza, Walkersville, MD, USA, Cat #. CC3162) up to passage 8. All cells were cultured at 37° C. in 5% CO₂ to 80% confluency before passaging. HUVECs and A549 cells were cultured in tissue culture polystyrene flasks coated with 0.1% gelatin (Sigma-Aldrich, Saint Louis, MO, USA, Cat #. G1890). HUVECs. and PMA-treated THP-1 cells were trypsinized using TrypLE™ Express (GIBCO, Life Technologies, Cat #. 12605-010) for 3 min at 37° C. and resuspended in EGM-2, DMEM with 10% FBS and 1% P/S and RPMI with 10% FBS and 1% P/S, respectively.

Regarding generation of macrophage-conditioned medium (Mφ-CM), one million THP-1 cells were first seeded per well of a 6-well plate containing 3 ml of RPMI-1640 medium with 10% FBS, 1% P/S, and 100 ng/ml of phorbol 12-myristate 13-acetate (PMA) (STEMCELL Technologies, Vancouver, Canada, Cat. #74042) to induce macrophage differentiation. After 24 hours, the attached cells were washed with phosphate-buffered saline (PBS) before changing to 3 ml of EGM-2 medium and incubated for 48 hours. Subsequently, the supernatant was extracted and filtered through a 0.22 μm filter before being diluted 1:1 with fresh EGM-2 medium. The Mφ-CM was stored at −80° C. and thawed right before use.

Regarding proteome profiler cytokine array, Mφ-CM was analyzed for its cytokine composition using the Proteome Profiler Human XL Cytokine Array Kit (R&D Systems, Minneapolis, USA, Cat. #ARY022B). 105 cytokines were simultaneously detected based on the manufacturer's instructions using antibodies and reagents from the kit. Signals in the cytokine array membrane were captured using the ChemiDoc™ MP Imaging System (Bio-Rad Laboratories, CA, USA, Cat. #17001402). To quantify the difference in the level of cytokines between control (unconditioned EGM-2 culture medium) and Mφ-CM, the average pixel intensity of each duplicate spot was measured using ImageJ v1.54f software. Cytokines were classified either as EMT inducers or EMT suppressors, and either as pro-inflammatory or anti-inflammatory cytokines, then plotted as a log 2 fold change of Mφ-CM to control using GraphPad Prism 9.4.1 software.

Regarding spheroid formation, 500 A549 cell spheroids were formed using Aggrewell™ 400 plates (STEMCELL Technologies, Cat. #34411) according to the manufacturer's instructions. Briefly, 500 μL of anti-adherence rinsing solution (STEMCELL Technologies, Cat. #07010) was added into each well and centrifuged at 1,300×g for 5 minutes to prevent cell adhesion. After removing the rinsing solution, 600,000 cells of A549 cells suspended in 2 ml of DMEM with 10% FBS and 1% P/S were added into each well to achieve the targeted 500 cell spheroids in each microwell. Cells were aggregated by centrifugation at 100×g for 3 minutes and were incubated for 3 days before being flushed out for subsequent experiments. As appreciated by a person skilled in the art, other target biological samples, e.g. spheroids of other cancer diseases are cultured for being observed by using the system in accordance with alternative embodiments of the present invention.

Regarding cell seeding for 2D culture, post-trypsinized A549 cells were seeded in tissue culture-treated 48-well plates at 15,000 cells/cm² and left overnight for attachment. After overnight incubation, the culture medium was replaced with either control (EGM-2 medium) or the following experimental media: (i) EGM-2 medium with 5 ng/ml of recombinant human TGF-β1 (PeproTech, New Jersey, USA, Cat. #100-21); (ii) EGM-2 with Mφ-CM; and (iii) EGM-2 with Mφ-CM and TGF-β1. This is followed by incubation for 2 days before further analysis.

Preferably, the system may further comprise an imaging device, such as a microscopic imager, adapted to capture images of the biological cells in the center channel to facilitate visualizing and recording intravasation events of the biological cells in the center channel. To analyse A549 cell morphology, phase contrast images of A549 were taken at with 100× objective with a Nikon ECLIPSE Ti2-A inversion fluorescence microscope (Nikon Instrument Inc.). Circularity of cells was assessed via ImageJ software. The circularity of 1.0 indicated a perfect circle while values approaching 0.0 represented a less circular or more elongated cell morphology.

Regarding viability assay, A549 cells subjected to different conditions were investigated for cellular viability by two approaches. First, the number of cells per field of view was counted based on nuclear staining by 4′,6-diamidino-2-phenylindole (DAPI). In addition, Live/Dead Assay was performed using Live/Dead viability/cytotoxicity kit (Invitrogen, Waltham, Massachusetts, USA, Cat. #L3224) according to the manufacturer's instructions. Dead and live cells were stained with 4 μM ethidium homodimer-1 and 2 μM calcein acetoxymethyl ester, respectively, for 15 minutes at 37° C., prior to imaging with a Nikon ECLIPSE Ti2-A inversion fluorescence microscope with 10× objective.

For immunocytochemistry of 2D cultures, a summary of the sources of antibodies and reagents used in this study is presented in Table 1. Cell fixation was first performed using 4% paraformaldehyde (PFA) followed by permeabilization with 0.1% Triton X-100 for 15 minutes and 1-hour blocking with 3% bovine serum albumin (BSA) (Sigma-Aldrich, Saint Louis, USA, Cat. #A7906). The cells were then incubated overnight with primary antibodies against E-cadherin and vimentin. Upon overnight incubation, cells were washed thrice with PBS and treated with Alexa Fluor 555-conjugated secondary antibodies for 1 hour followed by 10 minutes of DAPI incubation. Fluorescently stained E-cadherin and vimentin were visualized using Nikon ECLIPSE Ti2-A with 63× objective and the fluorescence intensity was calculated using ImageJ v1.54f software with the following formula:

Corrected total cell fluorescence (CTCF)=integrated density−(area of selected cell×mean background fluorescence intensity)

TABLE 1
List of antibodies and reagents for immunocytochemistry in 2D.

| Reagents | Dilution | Supplier | Cat. # |
|---|---|---|---|
| Mouse monoclonal anti-E-cadherin | 1:500 | Thermo | 13-1700 |
| | | Fisher | |
| Mouse monoclonal anti-Vimentin | 1:500 | Thermo | MA5-11883 |
| | | Fisher | |
| Goat anti-mouse-IgG AF 555 | 1:500 | Abcam | ab150118 |
| 4′,6-diamidino-2-phenylindole | 1:700 | Thermo | 62247 |
| (DAPI) | | Fisher | |

RNA extraction and cDNA synthesis were performed using RNAiso Plus (Takara Bio Inc., Cat. #9109) and PrimeScript™ RT Master Mix (Takara Bio Inc., Cat. #RR036A), respectively, following the manufacturer's protocols. For RT-qPCR analysis, 50 ng of cDNA and target-specific primers (Table 2) were amplified with ChamQ SYBR Color qPCR Master Mix (Vazyme, Cat. #Q411-03) on a QuantStudio™ 7 Pro Real-Time PCR System (Applied Biosystems™, Carlsbad, CA, USA). Gene expression levels were quantified using the cycle threshold (ΔΔCT) values which were normalized to housekeeping gene GAPDH and the relative fold change was compared to A549 control condition (EGM2 only) collected 24 hours after medium change. Another alternative housekeeping gene B2M was used to ensure the validity of GAPDH, which yielded similar results.

TABLE 2
Primer sequences used in RT-qPCR.

| Gene | Forward Primer | Reverse Primer |
|---|---|---|
| ZEB1 | 5′-GGCATACACCTA | 5′-TGGGCGGTGTA |
| | CTCAACTACGG-3′ | GAATCAGAGTC-3′ |
| | (SEQ ID No. 1) | (SEQ ID No. 2) |
| ZEB2 | 5′-AATGCACAGAG | 5′-CTGCTGATGTG |
| | TGTGGCAAGGC-3′ | CGAACTGTAGG-3′ |
| | (SEQ ID No. 3) | (SEQ ID No. 4) |
| SNAI1 | 5′-TGCCCTCAAGA | 5′-GGGACAGGAGA |
| | TGCACATCCGA-3′ | AGGGCTTCTC-3′ |
| | (SEQ ID No. 5) | (SEQ ID No. 6) |
| SNAI2 | 5′-ATCTGCGGCAA | 5′-GAGCCCTCAGA |
| | GGCGTTTTCCA-3′ | TTTGACCTGTC-3′ |
| | (SEQ ID No. 7) | (SEQ ID No. 8) |
| TWIST1 | 5′-GCCAGGTACAT | 5′-TCCATCCTCCA |
| | CGACTTCCTCT-3′ | GACCGAGAAGG-3′ |
| | (SEQ ID No. 9) | (SEQ ID No. 10) |
| B2M | 5′-CCGTGTGAACC | 5′-CCAATCCAAAT |
| | ATGTGACTT-3′ | GCGGCATCT-3′ |
| | (SEQ ID No. 11) | (SEQ ID No. 12) |
| GAPDH | 5′-CCAGGGCTGCT | 5′-ATTTCCATTGA |
| | TTTAACTCTGGTA | TGACAAGCTTCCC |
| | AAGTGG-3′ | GTTCTC-3′ |
| | (SEQ ID No. 13) | (SEQ ID No. 14) |

Regarding cell migration assay, A549 cells were cultured in 48-well plate wells until confluency. The culture medium in each well was first replaced with PBS, upon which the confluent layer of A549 cells was scratched using a P1000 pipette tip to create a gap. Floating cells were removed by washing with PBS, before the initial culture medium was added back.

Microscopic images of the center channel may be captured at a predetermined time interval. For example, phase contrast imaging was subsequently performed at 0-, 6-, and 12-hour time points using Nikon ECLIPSE Ti2-A inversion fluorescence microscope with 100× objective. The gap area at specific time points was quantified using ImageJ v1.54f software.

Regarding spheroid invasion assay, A549 spheroids were centrifuged and resuspended in different conditions at a density of 1,200 spheroids/ml. For each well of 48 well plate, 75 μL of spheroid suspension, which was estimated to contain 15 spheroids, was mixed one-to-one with 75 μL of 2 mg/ml neutralized collagen type I TeloCol®-6 hydrogel (Advanced BioMatrix, Carlsbad, USA, Cat. #5225). Collagen type I hydrogels embedded with spheroids were incubated for two hours at 37° C. to achieve polymerization before being overlaid with 300 μl of medium. EGM-2 medium was used as control. The spheroids were incubated for 24 hours to investigate their invasion ability in different conditions. After 4% PFA fixation for 30 minutes fixation, the spheroids were imaged using Nikon ECLIPSE Ti2-A inversion fluorescence microscope with 100× objective. To quantify the invasion ability of the spheroids, the area of invasion was manually traced and measured using ImageJ v1.54f software and divided by the initial spheroid area.

Regarding immunocytochemistry for 3D culture, after 4 days of culture, the devices were washed with PBS and the cultures fixed with 4% paraformaldehyde (PFA) (Thermo Scientific, Cat #. 5735) in PBS through the medium channels for 15 min, then permeabilized with 0.25% Triton X-100 in PBS for 10 min. After blocking with 5% BSA for 1 hour, samples were incubated with the respective primary antibodies (see Table 3) in PBS containing 0.5% BSA for 16 hours at 4° C. Samples were then washed three times with PBS for 5 minutes each, before being incubated with secondary antibodies (see Table 3) for at least 3 hours at room temperature. The samples were washed and stored in PBS at 4° C. before imaging.

TABLE 3
List of antibodies and reagents for immunocytochemistry in 3D.

| Reagents | Dilution | Supplier | Cat. # |
|---|---|---|---|
| Mouse monoclonal anti-VE-cadherin | 1:200 | Santa Cruz | Sc-9989 |
| Mouse monoclonal anti-Laminin | 1:200 | Abcam | ab77175 |
| Mouse monoclonal anti-CD31 | 1:200 | Abcam | ab9498 |
| Rabbit monoclonal anti-Vinculin | 1:200 | Abcam | ab155120 |
| Anti-mouse-AF-555 | 1:200 | Abcam | ab150118 |
| Anti-mouse-AF-488 | 1:200 | Abcam | ab150113 |
| Anti-rabbit-AF-647 | 1:200 | Abcam | ab150178 |
| Phalloidin-iFluor 647 | 1:700 | Abcam | ab176759 |
| Phalloidin-AF 555 | 1:700 | Abcam | ab176756 |
| 4′,6-diamidino-2-phenylindole | 1:700 | Thermo | 62247 |
| (DAPI) | | Fisher | |

Confocal imaging was performed using an inverted confocal microscope (Leica SP8, Leica Microsystems, Germany) with 20× and 63× objectives. Images of laminin, VE-cadherin and vinculin were taken and visualized. Orthogonal projections of the z-stack images were reconstructed to visualize events of cancer intravasation. All images were analyzed with ImageJ v1.54f software (imagej.nih.gov/ij/). Additionally, visualization of the MVNs in proximity of cancer spheroids was performed using Qiber3D

The system may further comprise a processor, preferably a machine-learning processing engine, arranged to analyze the captured images to identify cancer intravasation events in the biological cells based on extracted features from the images. Regarding quantification of MVN vascular junction and tubule length per field of view (FOV), phase contrast images (10×) of MVNs were analyzed using ImageJ v1.54f software and Angiogenesis Analyzer plugin 1.0 (imagej.nih.gov/ij/macros/toolsets/Angiogenesis%20Analyzer.txt). Briefly, raw images were converted into binary images using automated thresholds for binary tree analysis in the Angiogenesis Analyzer plugin. The number of junctions and total branching lengths were measured and presented as vascular junction number and tubule length per FOV, respectively. Junctions were denoted by points with at least 3 neighbors, and tubule length referred to the length of elements bound by two junctions or between one junction and one endpoint.

Regarding western blotting, PDMS devices were peeled off from the glass slide using a cutter and cells within the center channel were lysed using a 1:1 mixture of 2× Laemmli buffer and 2× protease inhibitor cocktail (Sigma-Aldrich, Cat #. P8340). Protein concentrations of collected samples were measured using a Bicinchoninic acid (BCA) Protein Assay Kit (Thermo Fisher, Cat #. A53226). Samples were denatured at 95° C. for 5 min and loaded at equal protein amounts into 8% SDS-polyacrylamide gels (Life Technologies, Cat #. HC2040) and subjected to electrophoresis at 120 V. After protein separation, samples were electrotransferred to a polyvinylidene difluoride membrane (Thermo Scientific, Cat #. 88,518) using a Power Blotter XL SYS (Life Technologies, Cat #0.34580). For membrane staining, membranes were incubated with 5% skimmed milk (Phygene Biotechnology Co Ltd, FuZhou, China, Cat #. PH1519) in TBS-Tween 20 (TBST), containing 50 mM Tris, 150 mM NaCl and 0.5% Tween 20 (Sigma-Aldrich, Cat #. P2287) to block non-specific antibody binding before incubation with primary antibodies (see Table 1) in TBST containing 1% skimmed milk at 4° C. overnight. After washing three times with TBST, secondary antibodies (see Table 4) resuspended in TBST containing 1% skimmed milk were added to the blots for 1 hour at room temperature. Proteins bands were then detected with ECL Super Signal West Pico Plus (Life Technologies, Cat #34,580) using ChemiDoc™ MP Imaging System (Bio-Rad) and quantified by Image Lab 6.1 software (Bio-Rad).

TABLE 4
List of antibodies and reagents for immunocytochemistry in 3D.

| Reagents | Dilution | Supplier | Cat. # |
|---|---|---|---|
| Mouse monoclonal anti-VE-cadherin | 1:500 | Santa Cruz | Sc-9989 |
| Mouse monoclonal anti-Laminin | 1:500 | Abcam | ab77175 |
| Anti-mouse-HRP | 1:5000 | Abcam | ab205719 |
| Anti-GAPDH | 1:500 | Abcam | ab181602 |

For live cell imaging, A549 and HUVECs were resuspended and pre-labelled with PKH26 Red Fluorescent and PKH67 Green Fluorescent Cell Linker Midi Kit for General Cell Membrane Labelling according to the manufacturer's protocol (Sigma-Aldrich, Cat. #MIDI26, MIDI67). The 24-hour live cell imaging was started on day 3 of culture and after the optional addition of an EMT-inducing cocktail, i.e. the reagent added to the media channels, to trace any events of cancer intravasation using a Leica Mica confocal microscope (Leica Mica, Leica Microsystems, Germany) with 20× objective. To quantify the rate of intravasation from the fluorescent images, the number of cancer cells localizing with the green fluorescence of MVNs was manually counted. Preferably, the EMT-inducing cocktail composite includes TGFb1 and a macrophage condition medium.

Regarding machine learning-assisted vessel segmentation and quantification of intravasation events, the inventors defined the localization of fluorescently labelled A549 cancer cells within the MVN as a proxy for cancer cell intravasation. All analyses were performed at the 24-hour time point of the fluorescent live cell images using Fiji (ImageJ v1.54f).

The images a may be processed by the following modules, including Vessel Segmentation, Tumor Spheroid Extraction and Segmentation Workflow.

Vessel segmentation was achieved through a machine learning-assisted approach on stacks of phase contrast and GFP channel images. This dual-channel combination enabled reliable segmentation even when GFP expression in HUVECs decreased during culture. Initially, the image was decomposited into individual stacks for thresholding. A default threshold was applied to the GFP and Cy3 channel to segment the HUVECs and Tumoroid. Thereafter, the tumoroid signal was subtracted and the images were recomposited with the phase channel. Subsequently, the Trainable Weka Segmentation (TWS) plugin was then trained to distinguish blood vessel area from non-blood vessel area using 28 images. Segmentation parameter settings include trainable features like Gaussian blur, Hessian, membrane projections, Sobel filter, and difference of Gaussians. The parameters are set as follows: membrane thickness of 1, patch size of 19, minimum sigma of 1.0, and maximum sigma of 16.0. The chosen classifier option is “fast random forest” using 200 trees and a batch size of 100. These settings determine how TWS learns and applies machine learning for image segmentation. Binary semantic segmentation was performed on images containing vessels and spheroids, with vessels classified as one class while spheroids and background were classified as another class. Thereafter, the binarized tumoroid fluorescent signal and blood vessel-positive regions were intersected to identify intravasated tumoroid-derived cells.

In Tumor Spheroid Extraction, each microscopy image used for quantification contained multiple tumor spheroids present on one microfluidic chip. A square ROI was drawn around each tumor region (tROI) to extract each spheroid separately. The Analyze Particles plugin was utilized to locate each spheroid, and the size of the tROI was set to 115% of the major axis after fitting an ellipse to the spheroid, ensuring only the signal in its immediate proximity was included for analysis.

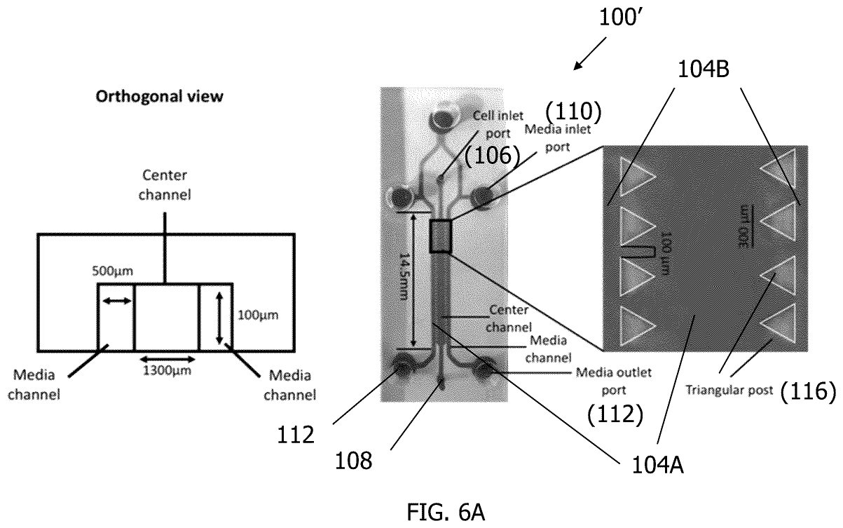

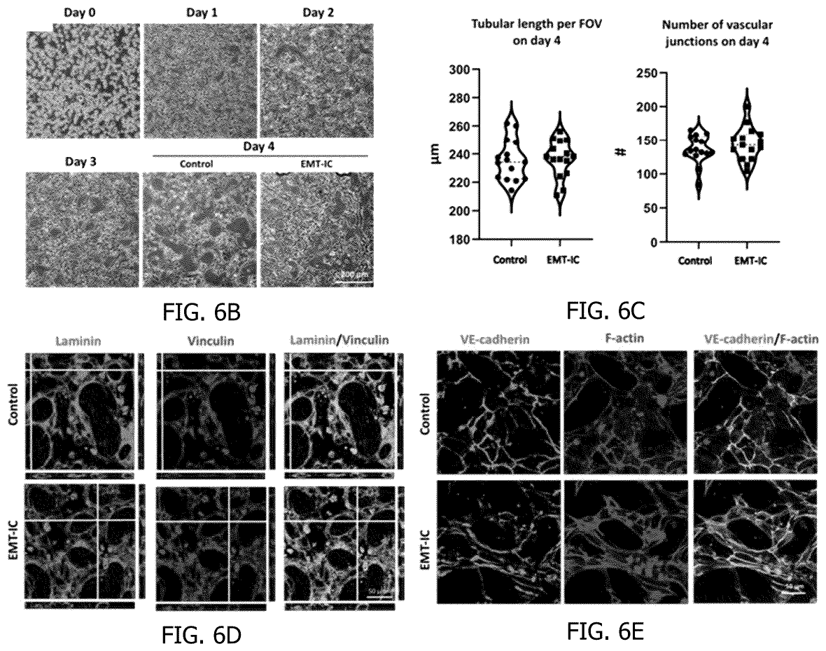

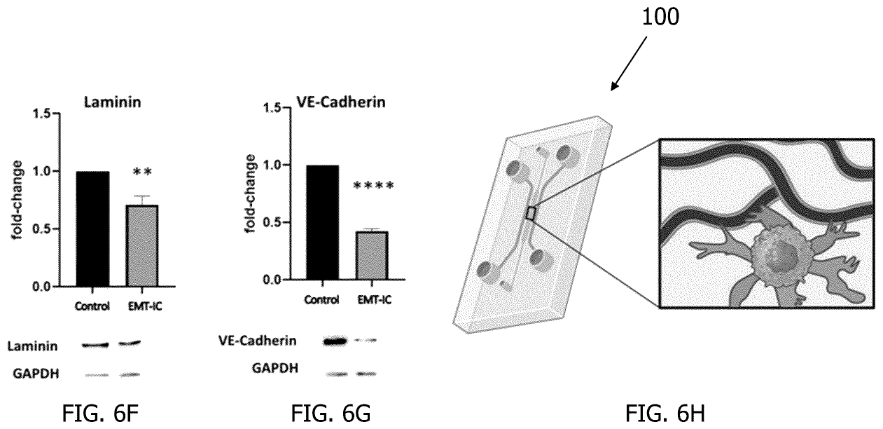

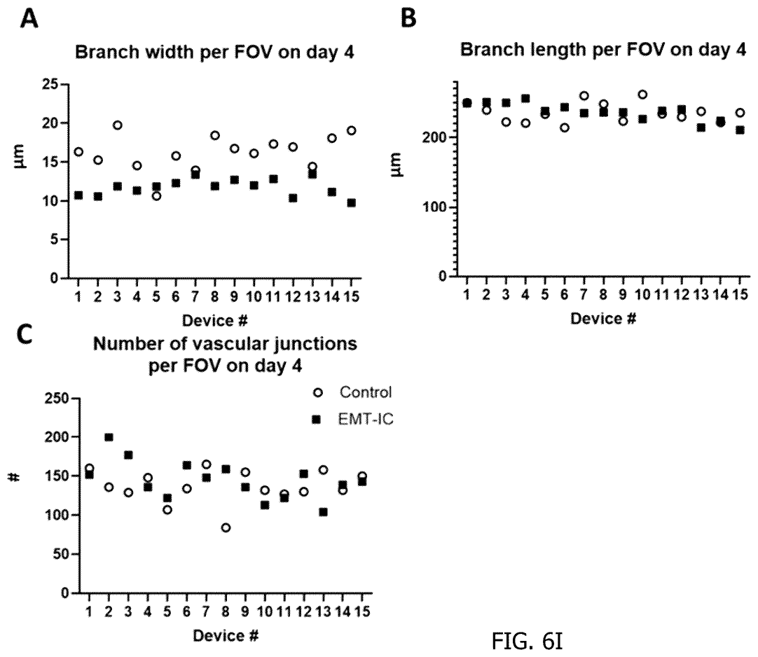

In Segmentation Workflow, MVNs were segmented by applying a default threshold to the GFP channel and subsequently applying the Weka classifier to the phase contrast and binarized GFP channels for each tROI (FIG. 6C). Morphological operations, such as Erosion and Dilation, were utilized to reduce imaging artifacts. The Analyze Particles plugin was then used to generate an ROI of the segmented vessels (vROI). This vROI was overlaid onto the channel containing the pre-fluorescently labeled A549 cancer cells. Fluorescence signals outside the vROI were excluded (FIG. 6C), and an Otsu threshold was applied to segment the cancer particles inside the vROI. The fluorescent particles of A549 cells inside the segmented vessels were counted (minimum size: 50 μm², circularity 0.5-1.0).

ImageJ scripts were developed to facilitate high-throughput automated image analysis and are available at: github.com/anna-jaey/FijiScriptToolbox?tab=readme-ov-file#particle-counting-and-area-ratio-quantification-in-multi-channel-fluorescence-images.

The inventors performed also statistical analysis. At least three independent biological runs with at least two replicates each were performed for each experiment. Levene's test was conducted to test for homogeneity of variances across samples. For parametric samples, unpaired t-test and one-way ANOVA followed by post hoc Tukey test with multiple comparisons was performed for samples with two and three or more conditions, respectively. On the other hand, non-parametric samples with two and three or more conditions were tested with Mann-Whitney test and Kruskal-Wallis test, respectively. Data were shown as means±standard deviation each containing at least three replicates, and statistical significance was set as follows: *p<0.05; **p<0.01; ***p<0.001; ****p<0.0001. All micrographs were quantified using ImageJ v1.53t software (https://imagej.nih.gov/ij/) and all analyses were performed using GraphPad Prism v9.3.1 (GraphPad Software, San Diego, CA, USA, www.graphpad.com).

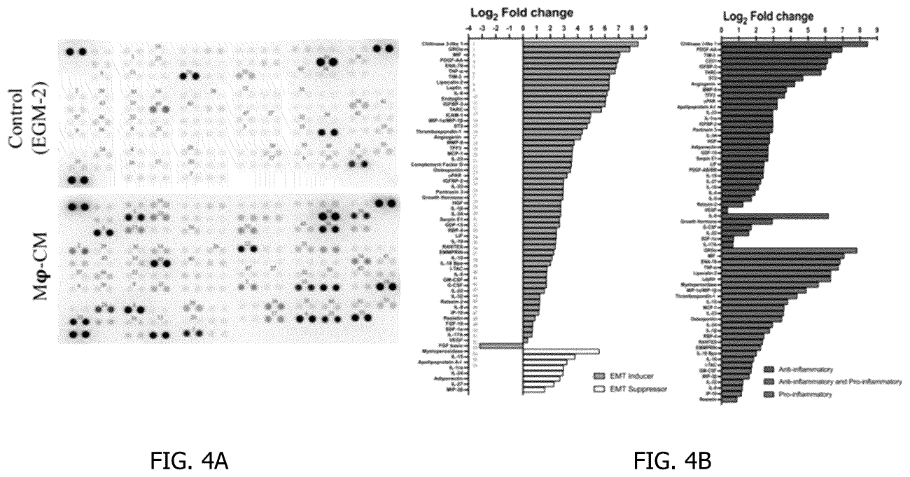

FIGS. 4A and 4B illustrate cytokine profiling of Mφ-CM by a proteome profiler array revealed an abundance of EMT-inducers secreted by THP-1-derived Mφ. With reference to FIGS. 4A and 4B, it is observed that Mφ-CM and TGF-β1 synergistically facilitate EMT in A549 cells. EMT is characterized by loss of apical-basal polarity, disruption of cell-to-cell adhesion and increased migration and invasion abilities of cancer cells into surrounding tissues. Standard EMT induction protocols utilize TGF-β1, as TGF-β1/SMAD serves as the EMT hallmark pathway resulting in the upregulation of EMT-related transcription factors. Nonetheless, the induction of EMT using a single growth factor often results in an incomplete transition and other EMT drivers have been considered. Physiologically, cellular components in the tumor microenvironment, such as CAFs and tumor-associated macrophages (TAM), have been implicated to have decisive roles in the EMT of cancer cells. Indeed, the secretome of TAMs was shown to contain a variety of cytokines that are involved in EMT induction, and macrophages or their conditioned medium have been demonstrated to promote EMT in epithelial cancer cells. Both, pro-inflammatory (M1) and immunomodulatory (M2) polarized macrophages have been implicated in driving EMT.

Adenocarcinoma human alveolar basal epithelial cells (A549) may be exposed to TGF-β1 and macrophage-conditioned medium (Mφ-CM) or the combination of both and investigated their ability to undergo EMT. Mφ-CM was derived from THP-1-derived macrophages. The composition of the Mφ-CM was analyzed using a proteome profiler array. Compared to the unconditioned EGM-2 culture medium, Mφ-CM was enriched in a wide array of known EMT-inducers, which outweighed the number of EMT-suppressors. At the same time the presence of pro-inflammatory and anti-inflammatory factors was relatively balanced, as shown in FIG. 4, suggesting Mφ-CM to contain a suitable cocktail for EMT induction in cancer cells.

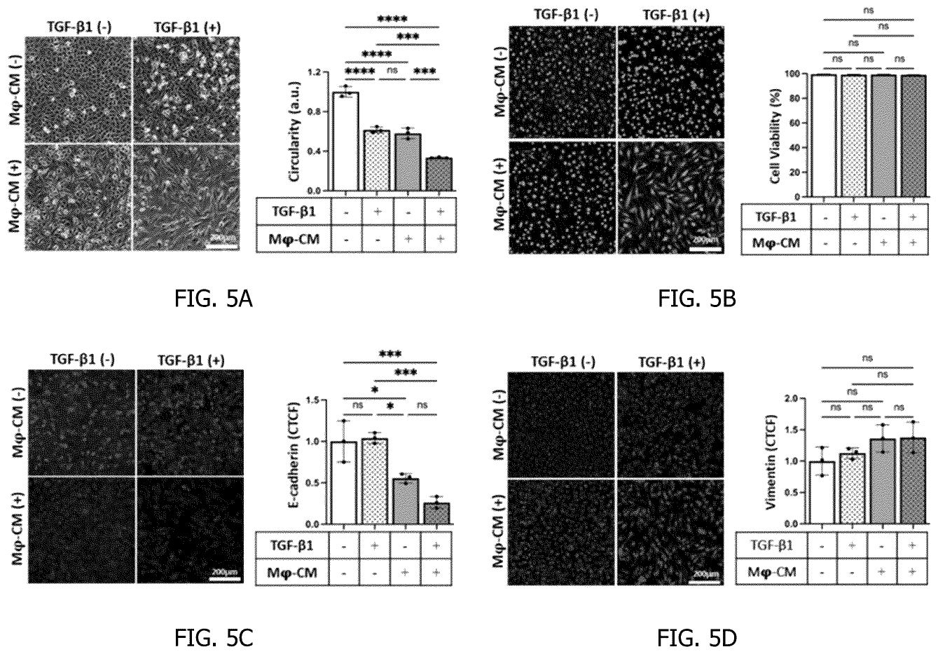

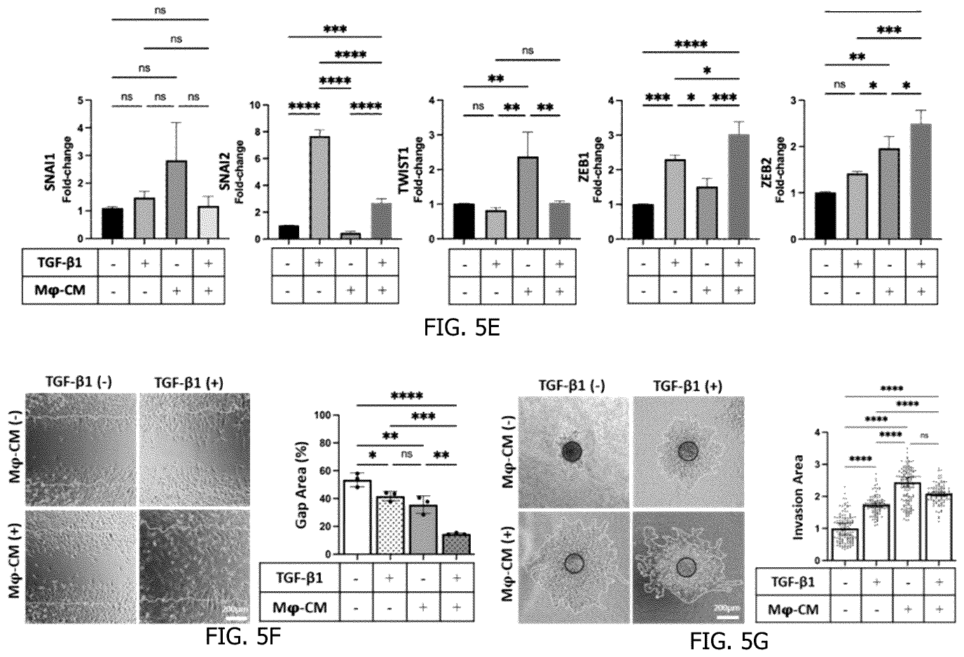

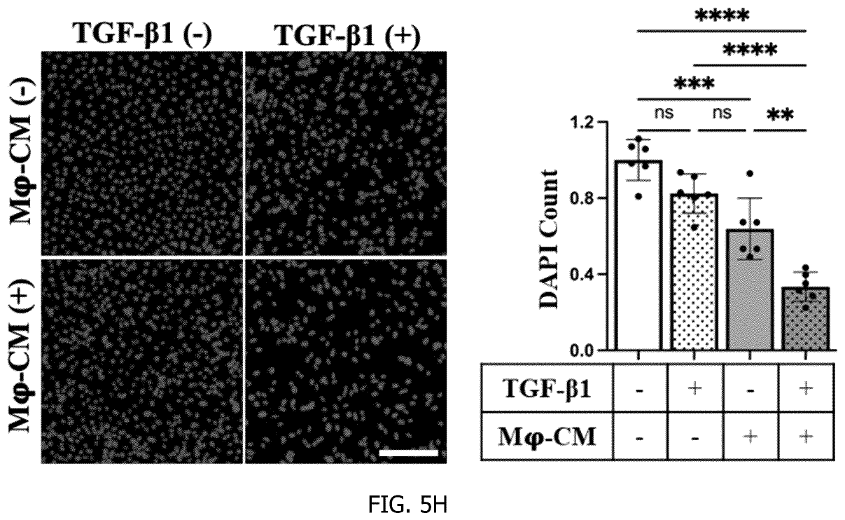

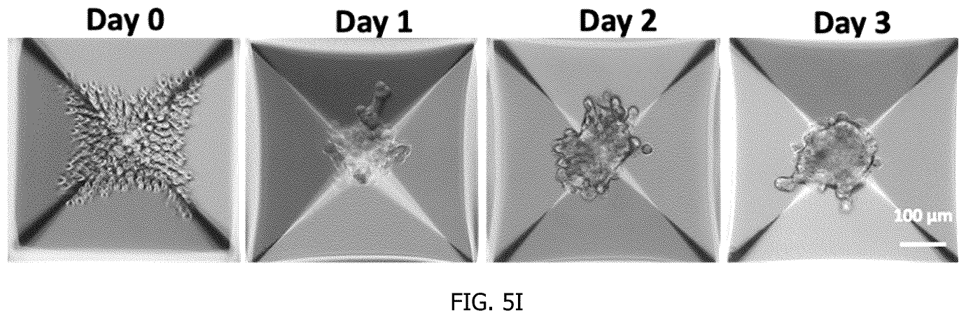

With reference to FIGS. 5A to 5G, A549 cells were thus incubated with the standard EMT-inducing cytokine TGF-β1, Mφ-CM, or a combination of both, to establish the best formulation of an EMT-inducing cocktail (EMT-IC). It can be observed that Mφ-CM and TGF-β1 synergistically drove EMT in A549 cells. In these Figures, *, p<0.05; **, p<0.01; ***, p<0.001; *, p<0.0001. n=3 biological replicates.

Morphologically, with reference to FIG. 5A, A549 cells exhibited a significantly decreased cellular circularity when exposed to TGF-β1 or Mφ-CM, while the combination of both induced the most observable change from cubic to spindle shape morphology. Referring to FIG. 5B, while none of the treatments affected cellular viability, the number of cells per field of view decreased as shown in FIG. 5H, which was attributed to the aforementioned changes of cell morphology to a larger spindle-like shape. Simultaneously, A549 cells exposed to TGF-β1 did not exhibit a visible decrease of epithelial marker E-cadherin, while A549 cells treated with Mφ-CM or the combination of both experienced the most significant reduction of E-cadherin signal, referring to FIG. 5C. An upregulation, albeit not significant, in mesenchymal marker vimentin was detected in A549 cells exposed to the combination of TGF-β1 or Mφ-CM referring to FIG. 5D.

Gene expression analysis revealed that an overall upregulation of key genes involved in EMT, when A549 cells were exposed to the combination of Mφ-CM and TGF-β1 as shown in FIG. 5E. Although SNAI1 gene expression exhibited a relatively high variability between treatment groups, SNAI2 was significantly upregulated in A549 treated with TGF-β1 alone or in combination with Mφ-CM. As shown in the Figure, TWIST1 was only significantly upregulated in A549 cells treated with Mφ-CM, while ZEB1 and 2 exhibited highest expression in A549 cells treated with the combination of both.

As marker expression and morphological changes are insufficient to confirm successful EMT, the functionality of the transformed A549 cells in terms of migration and invasion potential was investigated. Individually, TGF-β1 and Mφ-CM enhanced the migratory potential of A549 to a similar extent, while the combination of both more than doubled this effect, Referring to FIG. 2F. Next, A549 spheroids (tumor micromasses formed in microwells, referring to FIG. 5I, were embedded in 3D collagen type I hydrogels to measure the ability of tumor cells to invade into their local environment. Treatment with TGF-β1 increased their invasiveness, while treatment with Mφ-CM or the combination of both resulted in comparable and most advanced invasion into the surrounding environment, as shown in FIG. 5G. Taken together, the combination of TGF-β1 and Mφ-CM induced the most effective EMT in A549 cells and served as the EMT-IC in all subsequent experiments.

It is observable that EMT-IC did not adversely affect the stability of MVNs in microfluidic devices. Using an established three channel microfluidic device, HUVECs were seeded in fibrin hydrogels into the center channel, where they formed stable MVNs by vasculogenesis. With reference to FIG. 6A, by using an established three channel microfluidic device 100′, HUVECs were seeded in fibrin hydrogels into the center channel, where they formed stable MVNs by vasculogenesis. FIG. 6B to 6H illustrates the effect of EMT-IC (TGF-β1 & Mφ-CM) on the stability and integrity of MVNs. In these Figures, **, p<0.01; ****, p<0.0001. n=3 biological replicates.

Similar to the embodiment as shown in FIGS. 2 and 3, the microfluidic device 100′ in FIG. 6 is also a 3-channel microfluidic chip with a center channel 104A and a pair of media channels 104B sandwiching the center channel 104A. In addition, the center channel 104A is connected with a cell inlet 106 and a cell outlet 108, and each of the media channels 104B is connected to a media inlet 110 and a media outlet 112. In addition, the microfluidic device 100 comprises a plurality of partitioning structures 116 arranged to separate the center channel 104A from the media channel(s) 104B, and these partitioning structures are triangular in shape as shown in the Figure.

In this exemplary experiment, on day 3, the culture medium was changed to EMT-IC or control medium (unconditioned EGM-2 lacking TGF-β1), and MVNs were incubated for an additional 24 hours as shown in FIG. 6B. MVNs formed consistently and reproducibly across various devices and biologic repeats, ensuring reproducibility and robustness of the model as shown in FIG. 6I. The EMT-IC had no apparent effects on MVN stability, as shown in the quantification of the total length of tubular structures, as well as the number of vascular junctions, referring to FIG. 6C. Co-staining of major basement membrane component laminin and focal adhesion marker vinculin demonstrated that endothelial structures were enveloped in a tight sheath of basement membrane, suggesting that the EMT-IC had no adverse effects on the apical-basal polarity of microvessels, with cellular focal adhesions closely interacting with vascular basement membrane as shown in FIG. 6D. Co-staining of vessel-specific endothelial adhesion molecule VE-cadherin and F-actin enabled the visualization of cell-cell junctions respective to the overall structure of MVNs. Clearly outlined cell borders were visualized in both conditions, albeit appearing more tethered in MVNs incubated with EMT-IC, referring to FIG. 6E. Semi-quantification of laminin levels, as determined by western blotting, suggested a decrease of basement membrane levels in MVNs exposed to EMT-IC as shown in FIG. 6F, while western blotting for VE-cadherin revealed a 50% decrease in protein levels in MVNs exposed to EMT-IC as shown in FIG. 6G.

With reference to FIG. 6H, to visualize intravasation events in a microfluidic device 100, A549 spheroids may be co-seeded with HUVECs into hydrogel center channels of microfluidic devices, and MVNs are allowed to form for the first 3 days of culture. Subsequently, the medium is changed to EMT-IC or unconditioned EGM-2 (control), and cultures are incubated for another 24 hours.

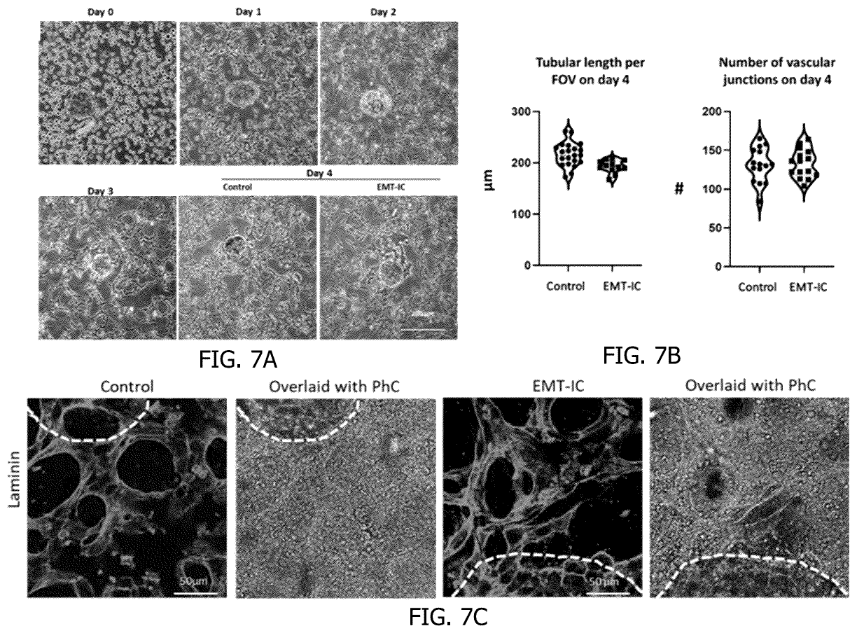

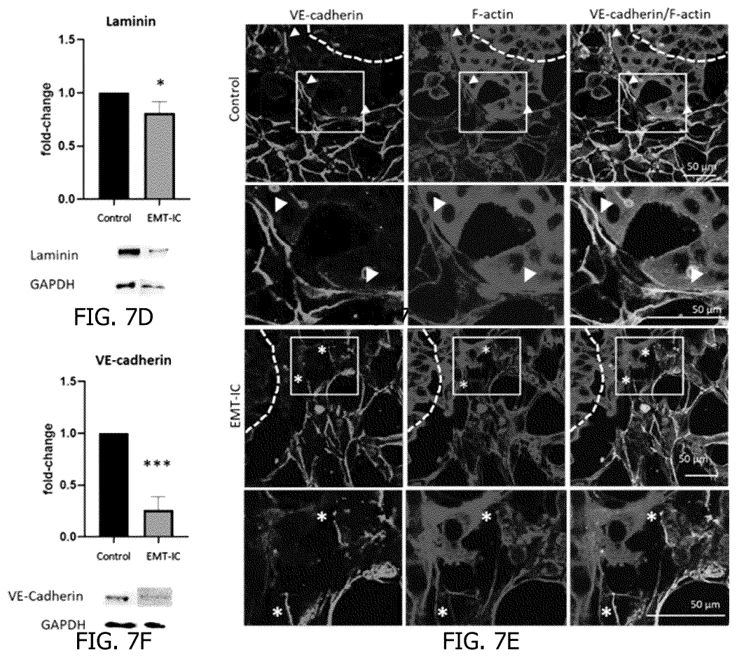

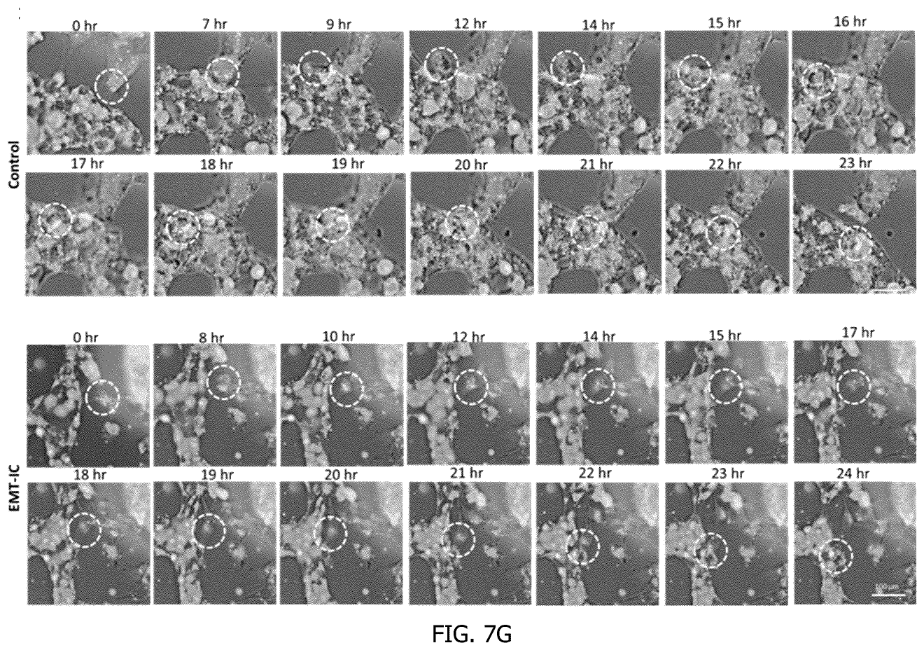

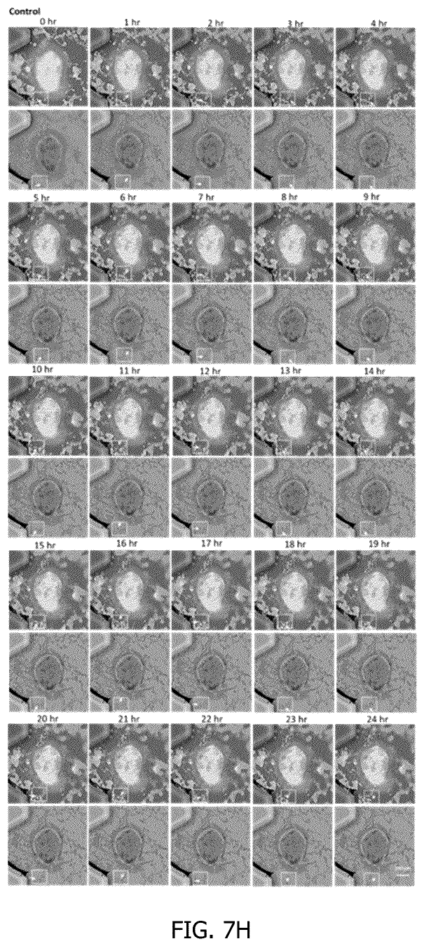

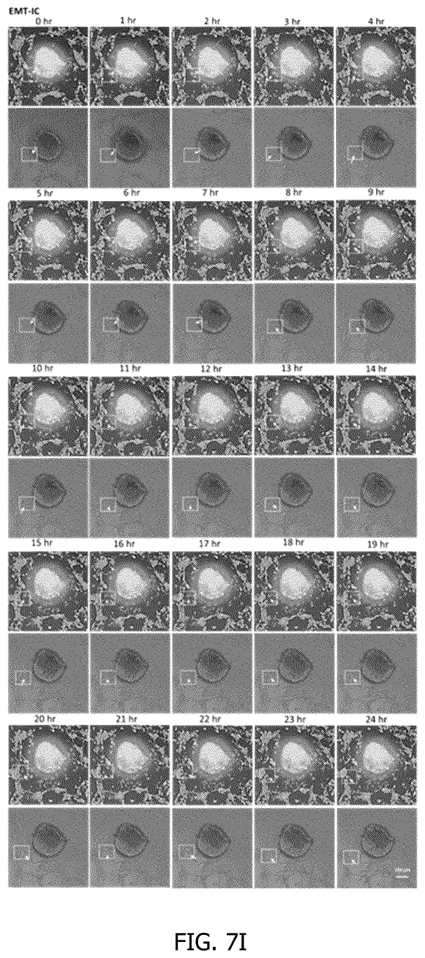

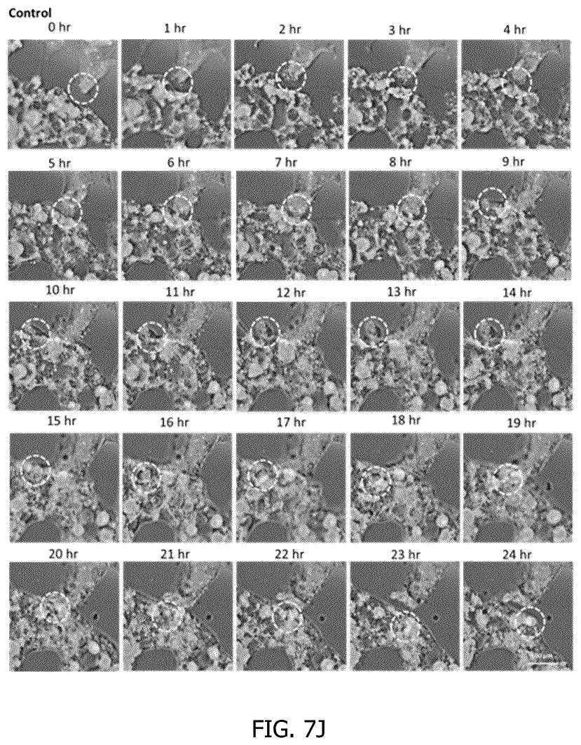

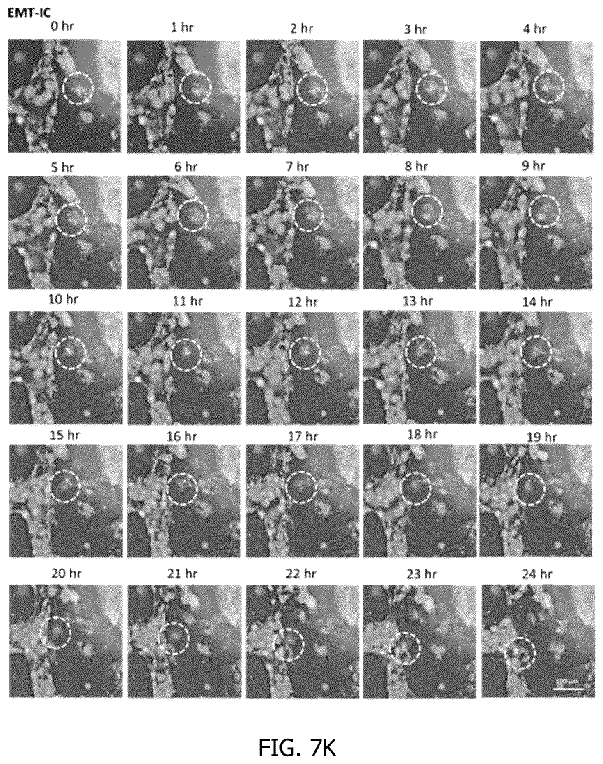

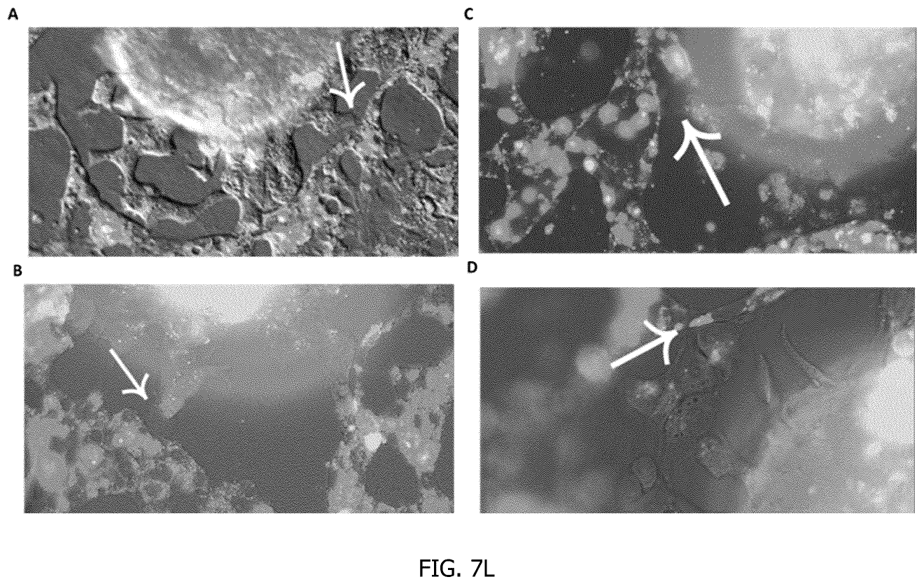

With reference to FIG. 7, it is observed that EMT-IC did not adversely affect the stability of MVNs when co-seeded with A549 spheroids in microfluidic devices. Evident from the phase contrast images, MVNs formed in close proximity around A549 spheroids, and were equally distributed throughout the chip, while tumor masses remained intact as shown in FIG. 7A. Referring to FIG. 7B, irrespective of the medium chosen, MVNs exhibited comparable stability on day 4. FIGS. 7A to 7G illustrates live cell fluorescent imaging of EMT-IC (TGF-β1 with Mφ-CM)-facilitated A549 cancer cell migration and intravasation into MVNs over 24 hours. In these Figures, n=11 biological replicates.

Referring to FIGS. 7C and 7D, immunostaining revealed that both MVNs and tumor masses synthesized laminin, while semi-quantitative evaluation of western blot analysis of co-cultures revealed a slight decrease in overall laminin protein levels. As expected, immunocytochemistry for VE-cadherin revealed that this cell adhesion molecule was restricted to MVNs, while F-actin was labelled in all cells in the co-culture. This allowed a clear visualization of cancer cells migrating out of the initial tumor masses and towards MVNs, referring to FIG. 7E. It is noteworthy that, in unconditioned medium, clear boundaries between migrating tumor cells and microvascular structures could be observed (FIG. 7E, arrowheads), while under EMT-IC, those boundaries were not apparent at sites of cancer cell-MVN interaction. Indeed, VE-cadherin appeared strongly downregulated at these specific locations (FIG. 7E, asterisks). Semi-quantification of western blot results for VE-cadherin demonstrated a more than 50% decrease in culture with EMT-IC as shown in FIG. 7F.

In addition, live cell imaging of fluorescently labelled A549 spheroids and MVNs was observed over 24 hours to trace events of intravasation into MVNs as shown in FIGS. 7G to 7I. As cells were labelled with a non-permanent membrane dye, it did not outline the whole cell body after several days of culture. Hence, fluorescent labels were used as indicators of cell identity, while phase contrast (PhC) images were necessary to confidently track the movement of cells. Live cell imaging was performed at 10× to track the movement of all cells from a spheroid as shown in FIGS. 7H and 7I, while time-frames of observed intravasation events were presented in enlarged close-ups as shown in FIGS. 7G, 7J, 7K, and 7L. Overlays of fluorescent images and phase contrast allowed to observe cancer cells moving away from cancer spheroid, towards and along vessel walls. At sites of intravasation, cancer cells extended membrane protrusions through the microvascular wall, then maneuvered their bodies through the openings into MVNs (FIG. 7G, dashed circles). This was observed in control as well as EMT-IC conditions.

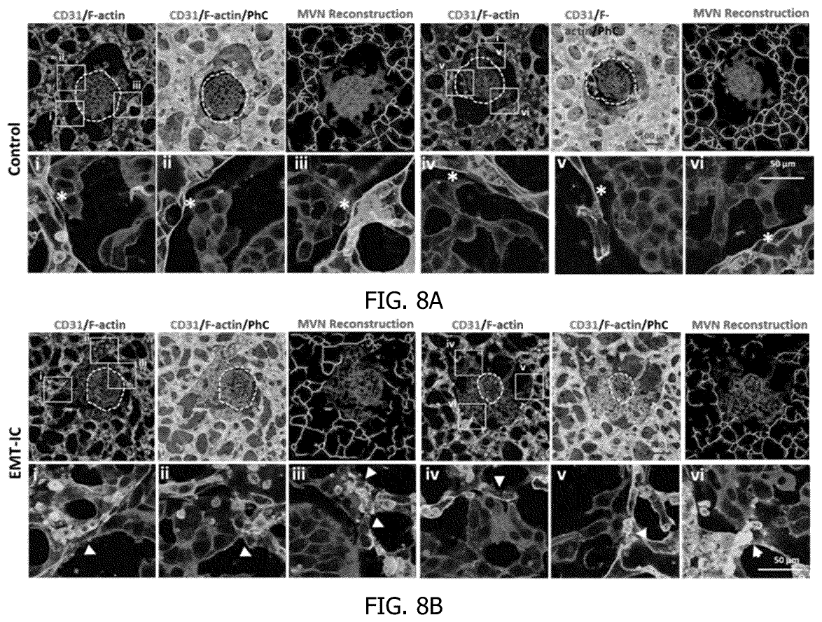

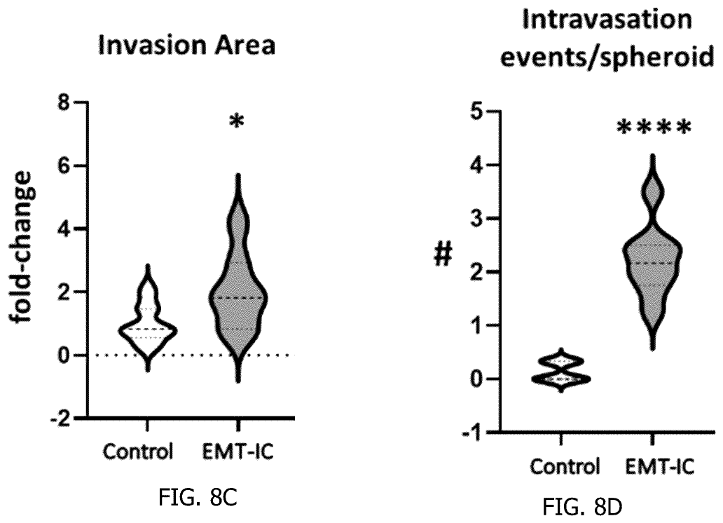

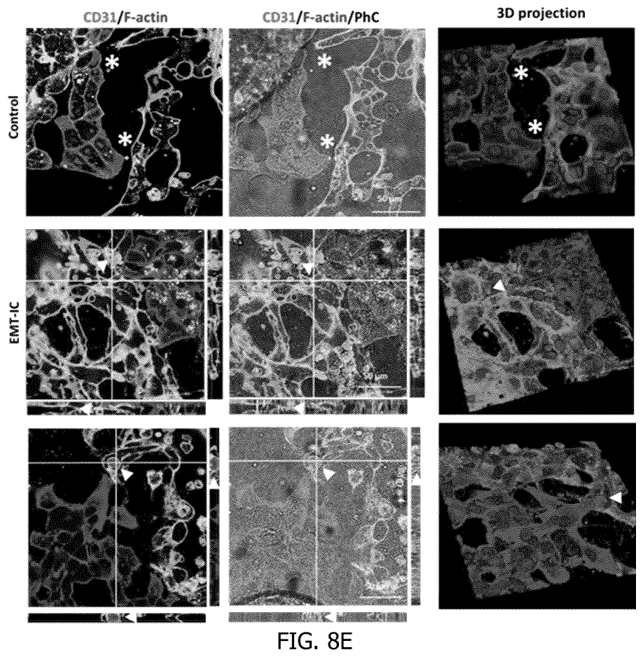

With reference to FIGS. 8A to 8E, it is observable that EMT-IC facilitated cancer cell intravasation into MVNs. To visualize intravasation events, day 4 co-cultures of A549 spheroids and MVNs were co-stained for endothelial marker CD31 and F-actin. In these Figures, *, p<0.05, ****, p<0.0001. Control=19 spheroids; EMT-IC=21 spheroids collected from 3 biological replicates.

To visualize intravasation events in more detail, after 24 h of exposure to control medium or EMT-IC, co-cultures of A549 spheroids and MVNs were fixed and co-stained for endothelial marker CD31 and F-actin. A549 exhibited a stronger fluorescent intensity for F-actin staining, enabling them to be distinguished from the CD31-positive, F-actin-weaker stained MVNs. As expected, cancer cells migrated out of A549 spheroids (initial tumor masses are indicated as white dashed lines) under both conditions as shown in FIGS. 8A and 8B. Nonetheless, semi-quantification of invaded area by migrating cancer cells demonstrated that cancer cells exposed to EMT-IC exhibited an increased invasion potential in fibrin hydrogels within microfluidic devices as shown in FIG. 8C. This was comparable to observations made in collagen I hydrogels previously, referring also to FIG. 5G.

In control conditions, close-ups of confocal microscopy images (FIGS. 8A and 8B white solid rectangular frames) revealed that although cancer cells were in proximity of microvessels, commonly clear borders remained visible (FIG. 8A, white asterisks), suggesting that no intravasation events took place. In contrast, under EMT-IC, migratory cancer cells extended membrane protrusions into microvascular structures (FIG. 8B, arrowheads), thus initiating intravasation. Manual quantification of intravasation events, as defined in FIG. 8B, indicated an average of two intravasation events per spheroid when exposed to EMT-IC.

In contrast, referring to FIG. 8D, intravasation events were rare (<1 intravasation event/spheroid) under control conditions. This was further confirmed by high-resolution confocal microscopy, where clear separations between invading cancer cells and MVNs were mainly observed under control conditions (FIG. 8E, white asterisks). Under EMT-IC, cancer cell membrane protrusions invaded microvascular structures, followed by maneuvering their cell bodies into the MVN lumen (FIG. 8E, arrowheads). The latter was particular evident, as cancer cell bodies appear surrounded by CD31 stained lumen in orthogonal cross-sections and in 3D projections (FIG. 8E, last row).

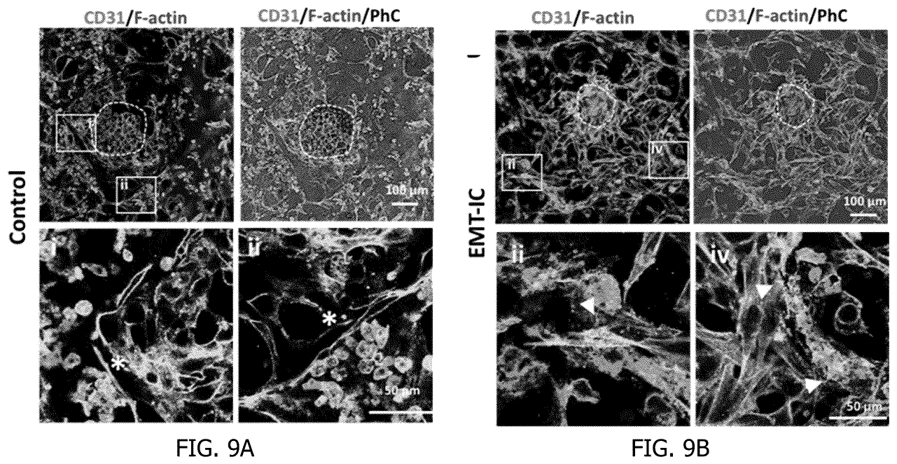

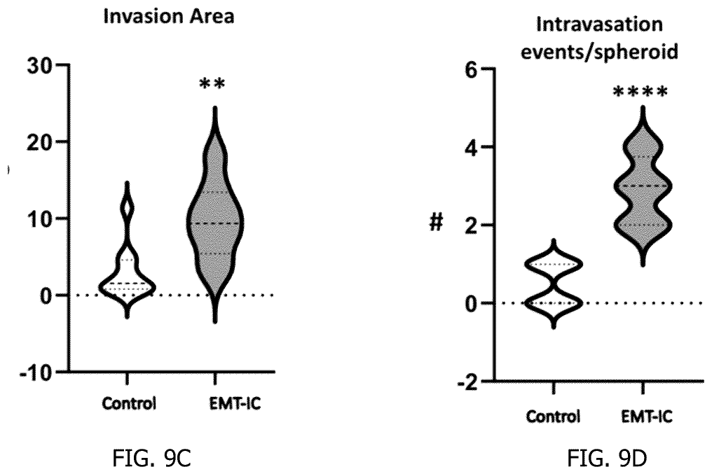

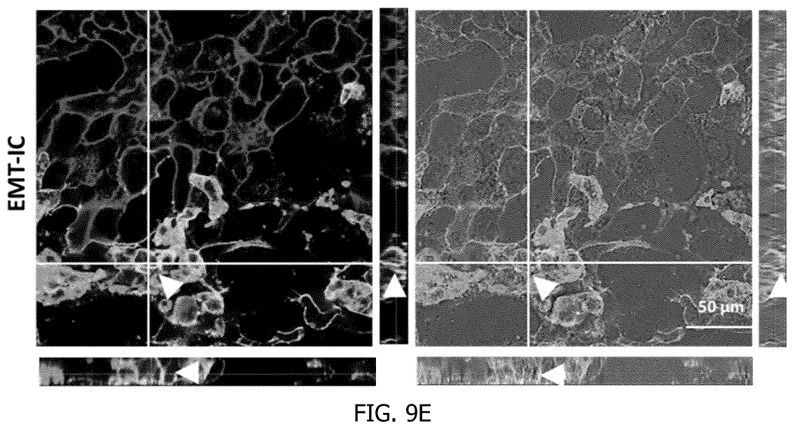

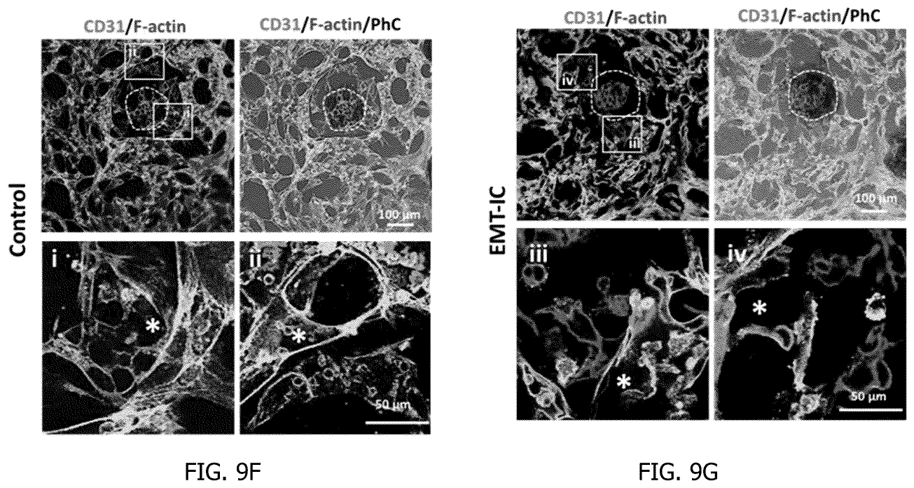

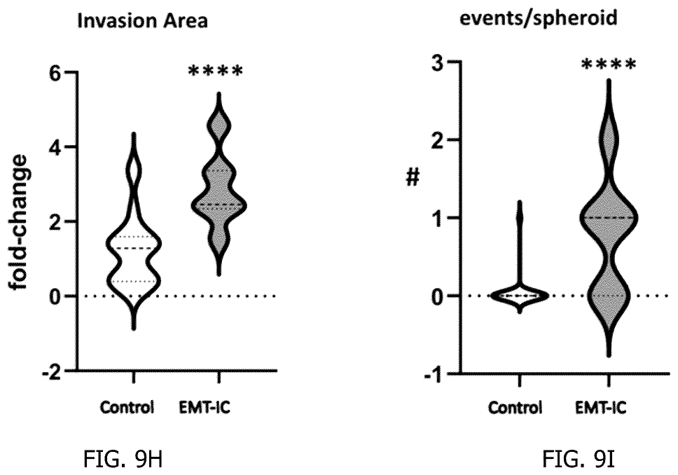

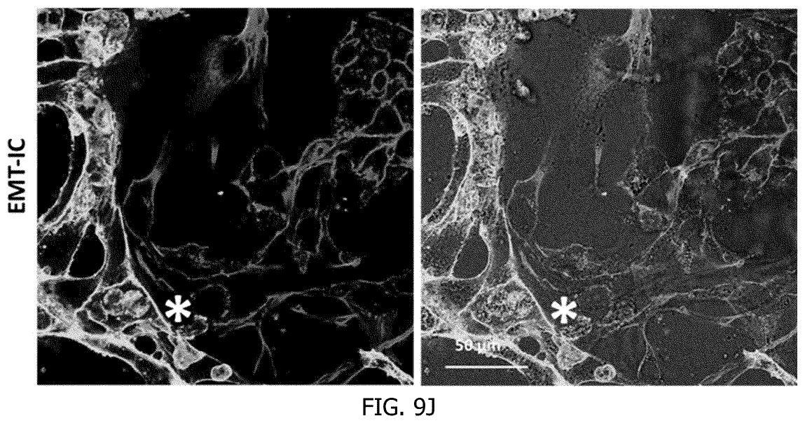

With reference to FIGS. 9A to 9J, in order to validate the universal applicability of the established lung cancer intravasation-on-a-chip model, two additional lung cancer cell lines: the non-metastatic BEAS-2B and the metastatic NCI-H1975 were introduced into the model. In these figure, **, p<0.01 and ****, p<0.0001. NCI-H1975: Control=10 spheroids; EMT-IC=8 spheroids collected from 3 biological replicates. BEAS-2B: Control=22 spheroids; EMT-IC=17 spheroids collected from 3 biological replicates.

Referring to FIGS. 9A to 9C, even under control conditions, the more aggressive metastatic NCI-H1975 cell line exhibited strong invasion potential, which could be extensively enhanced (10-fold) by EMT-IC. Congruently, NCI-H1975 cells were observed to frequently intravasate into the surrounding microvasculature under control conditions (0.5 events/spheroid), which could be substantially augmented by EMT-IC supplementation (3 events/spheroid), as shown in FIG. 9D. Successful intravasation of NCI-H1975 cells under EMT-IC was confirmed in high resolution, where cancer cells were identified within the microvascular lumen as shown in FIG. 9E.

In comparison, the response of BEAS-2B cells was substantially attenuated, as they invaded a qualitatively smaller area, which could only be slightly enhanced by EMT-IC (2-fold increase) as shown in FIGS. 9F to 9H. Furthermore, BEAS-2B cells did not exhibit any significant intravasation potential under control conditions, which could only be slightly elevated by EMT-IC, thereby maintaining baseline intravasation levels of less than 1 event/spheroid as shown in FIG. 9I, corresponding to their non-metastatic nature. High resolution microscopy confirmed overall well defined boundaries between BEAS-2B cancer cells and MVNs, even under EMT-IC, as shown in FIG. 9J.

With reference also to FIG. 4G to 4L, live cell imaging of fluorescently A549 spheroids and MVNs was observed over 24 hours to trace events of intravasation into MVNs. Any co-localization of red-labelled A549 cells within green-labelled MVNs was defined as an intravasation event. Live cell imaging confirmed augmented cancer cell shedding and migration, as well as an increased rate of A549 cells approaching the neighboring MVNs via extension of their pseudopodia in the presence of EMT-IC.

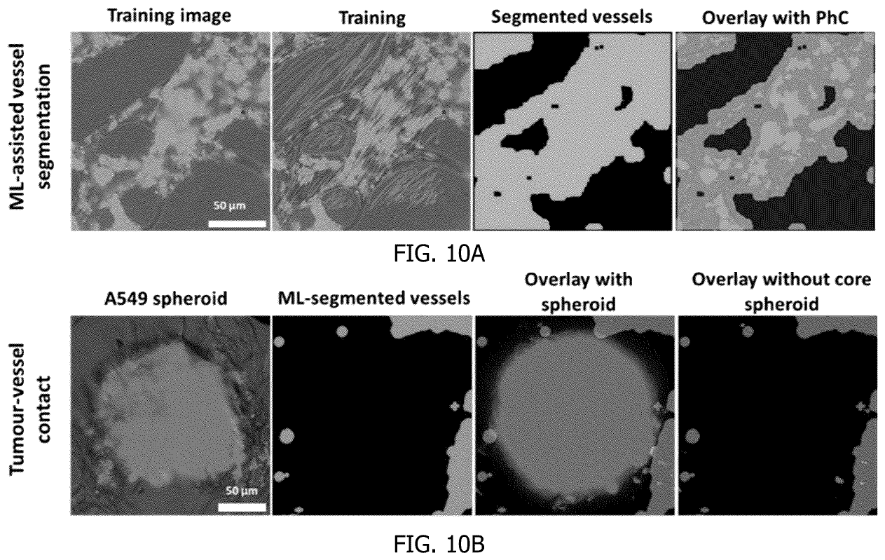

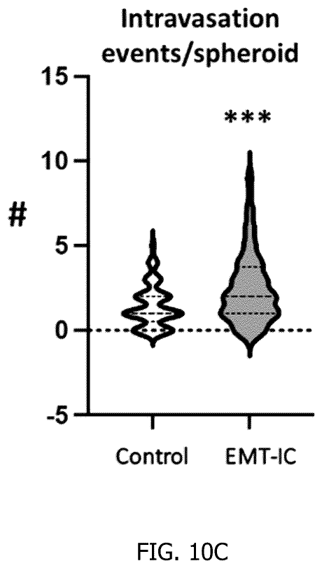

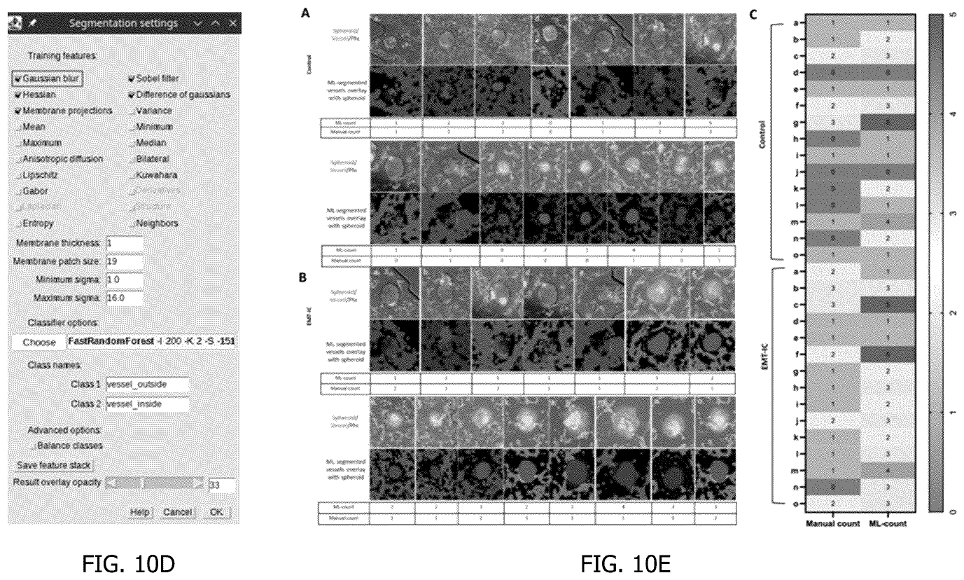

An image analysis workflow was developed to segment vessels and cancer cells and analyze their co-localization to quantify intravasation events in an unbiased manner. Using the TWS plugin, as shown in FIG. 10D, the inventors reliably segmented the MVNs around the cancer spheroids. Combined with threshold-based segmentation of the cancer spheroids, fluorescently labelled cancer particles within the segmented vessel area as a proxy for intravasation events were counted, referring to FIGS. 10A and 10B. The automated method showed similar trends to manual counting with reference to FIGS. 8D and 10E. It can be observed that, on average, three intravasation events per spheroid when exposed to EMT-inducing conditions (EMT-IC) and one intravasation event per spheroid under control conditions, referring to FIG. 10C. The total number of events per group was slightly increased with the automated workflow.

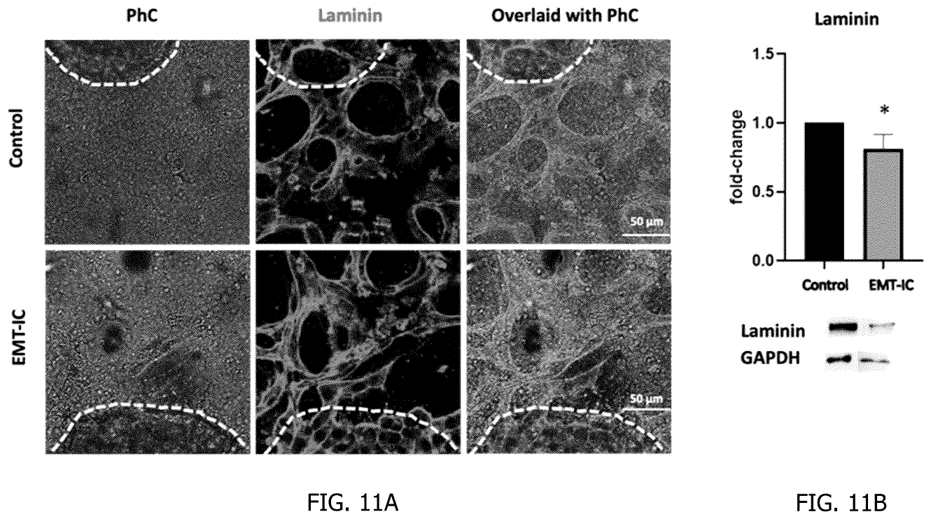

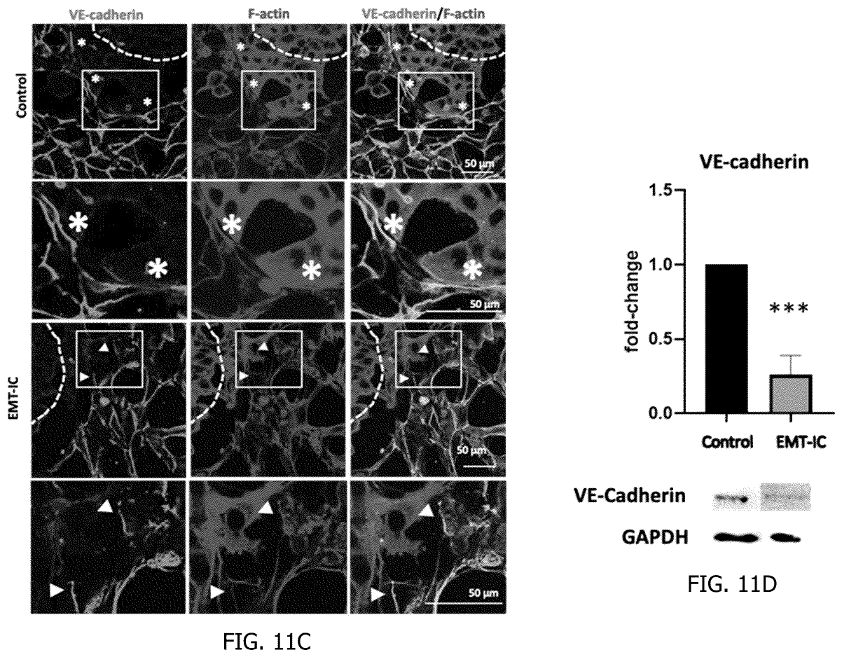

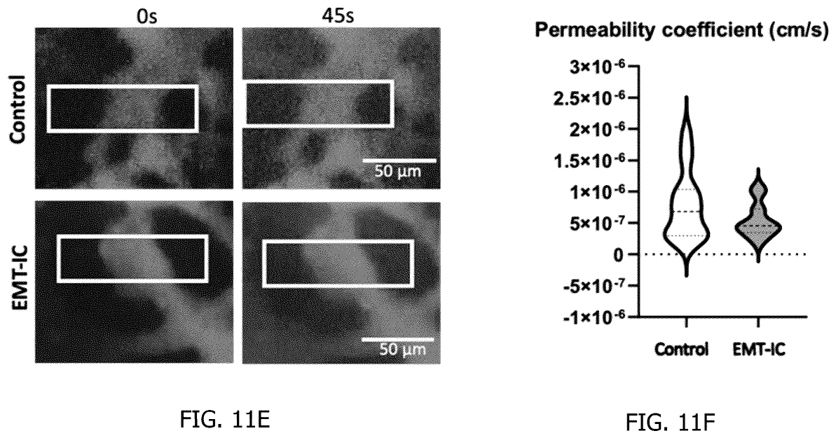

With reference to FIGS. 11A to 11F, there is shown the effect of EMT-IC (TGF-β1 with Mφ-CM) on the stability and integrity of MVNs in co-culture with A549 spheroids. In these Figures, *, p<0.05; ***, p<0.001, n=3 biological replicates.

Immunostaining of MVNs and A549 spheroids on day 4 revealed that both MVNs and tumor masses (identified as dense cell masses in phase contrast (PhC) images) synthesized laminin as shown in FIG. 9A, while semi-quantitative evaluation of western blot analysis of co-cultures revealed a slight decrease in overall laminin protein levels under EMT-IC as shown in FIG. 9B. As expected, immunocytochemistry for VE-cadherin revealed that this cell adhesion molecule was restricted to MVNs, while F-actin was labelled in all cells in the co-culture. Nonetheless, A549 exhibited stronger fluorescent intensity for F-actin, enabling them to be distinguished from MVNs. This allowed a clear visualization of cancer cells migrating out of the initial tumor masses and towards MVNs as shown in FIG. 11C. It is noteworthy that, in unconditioned medium, clear boundaries between migrating tumor cells and microvascular structures could be observed (FIG. 11C, asterisks), while under EMT-IC, those boundaries were not apparent at sites of cancer cell-MVN interaction. Indeed, VE-cadherin appeared downregulated at these specific locations (FIG. 11C, arrowheads). Semi-quantification of western blot results for VE-cadherin demonstrated a more than 50% decrease in culture with EMT-IC as shown in FIG. 11D.

As vascular barrier functions, characterized by low vascular permeability, rely on tightly connected cell-cell junctions and a well-structured basement membrane, vascular wall permeability was next assessed. For this HUVECs were seeded into the medium channels of the cancer intravasation-on-a-chip on day 2 of culture to form an additional continuous endothelial monolayer at the media-hydrogel interface. By day 4, these endothelial cells had anastomosed with the MVN in the center channel, creating vascular openings that connected to the medium channels.

To assess perfusion capability, FITC-PVP (40 kDa) was introduced together with EMT-IC into one of the medium channels, and live fluorescence microscopy demonstrated that MVNs were perfusable from the medium channels under both conditions as shown in FIG. 11E. Next, vascular permeability was evaluated by measuring the diffusive flux of solutes across the vessel wall. Specifically, the transport of FITC-PVP across the microvascular wall was quantified by tracking changes in fluorescence intensity within a defined perivascular (hydrogel) region over time (imaging every 15 seconds for 15 minutes). Assuming the microvessels formed circular tubular structures with diameters below 50 μm, the permeability coefficient was calculated using established methods.

Under initial exposure to EMT-IC, the MVNs exhibited a permeability coefficient of 7.79±2.7×10⁻⁷ cm/s, which is comparable to that in control which has a permeability coefficient of 5.41±5.37×10⁻⁷ cm/s as shown in FIG. 11F. Measurements of vascular permeability after 24 h of exposure to EMT-IC were not possible, as vessel openings to media channels had closed at this point of the experiment under EMT-IC, while MVNs under control conditions remained perfusable.

These embodiments provide a microphysiological in vitro model of EMT-driven lung cancer intravasation coupled with ML-assisted image processing, enabling automated and unbiased quantification of intravasation events. This platform technology enables the visualization and investigation of underlying biological processes in high spatio-temporal resolution and its potential utilization as a drug screening tool. The universal applicability and sensitivity of the lung cancer intravasation-on-a-chip was demonstrated by the functional incorporation of various metastatic and non-metastatic lung cancer cell lines. Importantly, the baseline metastatic potential of cancer cell lines was clearly reflected in the measurements of invasion and intravasation potential in the devices, suggesting that the established model allows to predict cell specific metastatic potential and responsiveness to EMT induction, thus paving the way for testing of patient-specific samples.

Advantageously, the model is beneficial in its accelerated intravasation timeline, as live cell imaging data confirmed that intravasation happens frequently within the first hours upon EMT induction. The 24-hour endpoint thus provides a snapshot of actively intravasating cells, while early-intravasated cells are less likely to have migrated beyond the imaging field through the MVN. Importantly, the established system maintains excellent reproducibility within these first 24 hours. This compressed timeline represents a significant advantage over other alternative in vitro models and in vivo systems, particularly for high-throughput applications.

Moreover, a novel EMT induction cocktail based on the synergistic effects of macrophage conditioned medium and TGF-β was established, inducing a robust migratory and invasive behaviour in lung cancer cells, outperforming standard EMT induction methods. As both M1 and M2 polarized macrophages were implicated in facilitating EMT in cancer cells, the inventors opted for unpolarized macrophages, which can secrete pro- and anti-inflammatory factors, and decided on moving forward with their conditioned medium, based on the abundance of EMT-inducers within. The Mφ-CM contained a number of EMT inducers, including CHI3L1, IL-10, IL-6, IL-8, and TNF-α, which are known to play a role in cancer metastasis, migration, invasion, chemotaxis, and endothelial cell junction retraction. The complexity of cytokines in the inventors' model more closely resembled the heterogeneous tumor microenvironment in advanced NSCLC, making it suitable for studying EMT-induced cancer intravasation mechanisms.

The inventors investigated successful EMT using various markers on protein and gene expression levels. Indeed, the EMT process is controlled by multiple transcription factors, including SNAI1, SNAI2, ZEB1, TWIST, CarB-box-binding factor, Mesenchyme Forkhead 1, and Kruppel-like factor. These transcriptional regulators are modulated by intricate signaling networks within the tumor microenvironment, particularly through pathways involving TGF-β, Notch, and Wnt signaling. Notably, the expression of these factors follows a temporal hierarchy during EMT progression: SNAI1 activation initiates the transition, while subsequent induction of SNAI2, ZEB1, and TWIST serves to sustain the resulting migratory phenotype. Especially, ZEB1 and ZEB2 exhibited strongest upregulation in the EMT-IC condition, surpassing the effects of TGF-β1 alone. This is particularly significant as ZEB1 and 2 are linked to aggressive, stem-like phenotypes and immune evasion. The synergy between Mφ-CM and TGF-β1 on ZEB1 and 2 suggests that cancer cells adopted a more aggressive metastatic phenotype.

Besides marker expression the inventors also confirmed successful EMT on a functional level, where Mφ-CM and TGF-β1 synergistically promoted migratory and invasive potential of cancer cells. Indeed, Mφ-CM performed even better than TGF-β1, suggesting that the intrinsic biocomplexity of Mφ-CM is advantageous for inducing EMT in A549 cancer cells. It is noteworthy that even though the supernatant of macrophages has been reported to induce EMT in colon cancer cells, the synergistic effect of TGF-β1 and Mφ-CM for EMT induction has been investigated. Since the combination of TGF-β1 with Mφ-CM appeared to act synergistically to facilitate EMT in A549 cells based on their marker expression, as well as functionality, this combination was chosen as EMT-IC for the microphysiological model.

In addition, the EMT-IC had a direct effect on MVNs, reducing their basement membrane and cell-cell junction proteins, respectively, thus potentially making them more permissive to intravasating cells, while not affecting their overall stability or vascular barrier functions to small solutes in the initial time frame investigated. Nonetheless, the effects of longer incubation times with EMT-IC were not tested.

The enhanced cancer cell invasion potential under EMT-IC, as observed in collagen type I hydrogels, was also reproduced in co-cultures with MVNs in fibrin hydrogels in microfluidic devices, suggesting that, advantageously, A549 cells, as well as other lung cancer cell lines underwent functional EMT in this co-culture set-up, albeit based on their metastatic predisposition. Advantageously, initiation of intravasation was mainly observed in co-cultures upon exposure to EMT-IC in metastatic lung cancer cell lines, where intravasation events were defined as cancer cells inserting membrane protrusions into microvascular structures. Besides the systemic effect of EMT-IC on vascular junctions, decrease in VE-cadherin was also specifically seen at sites of cancer cell entry. This suggests a direct communication of EM-transformed cancer cells and endothelial cells, resulting in permissive entry points in the microvessel walls. With the established microphysiological model, these interactions can be visualized and studied under high spatio-temporal resolution, as well as investigated for potential drug targets for anti-cancer treatments.

Various microphysiological systems of cancer metastasis exist. They mainly focus on later stages of cancer metastasis, such as extravasation events. It should be pointed out that physiologically representative models of cancer intravasation are worthwhile to focus on, as EMT-driven cancer intravasation serves as the rate-limiting step for circulating cancer cells. Targeting the early stages of the metastasis cascade is thus likely more effective in preventing cancer cells from spreading.

The inventors devised that a limitation of in vitro cancer metastatic research is the time-consuming manual quantification of the rate of cancer intravasation. For example, a similar study used a ‘thin plate spline’ method to define tissue boundaries and compartment across multiple organ-on-a-chip models for quantifying tumor intravasation events. Such mathematical modelling was required owing to variations that originate during organoid-on-a-chip manufacturing and experimental workflow. Automated image quantification utilizing ML-based approaches offers a solution, enabling unbiased analysis of large-scale data in various formats for tumor staging, cancer susceptibility, recurrence, and patient survival predictions.

Advantageously, an image analysis workflow using readily available, non-commercial and open-source tools to quantify intravasation events in a reliable and efficient manner has been developed. By defining intravasation events as the localization of fluorescently labelled cancer particles within segmented MVN areas, the inventors' automated method showed trends consistent with manual counting, underlining its reliability. Notably, in accordance with embodiments of the present invention, the ML-assisted workflow detected, on average, one more intravasation event per sample than manual counting. This discrepancy may be due to the enhanced capabilities of ML-based analysis detecting intravasation events missed by the human eye. In addition, the image analysis workflow in accordance with the embodiments of the present invention is universally applicable as it utilizes fluorescence signal overlap between tumor spheroids and endothelial cells to delineate intravasation events. The inventors observed that quantification of intravasation events was similar to manual analyses and was robust throughout multiple biological runs.

In addition, ML-based tools may be used in cancer research for staging, prognosis, extravasation, and lymph node metastasis. Future advancements combining physiologically relevant in vitro models, with 3D microscopy, 3D image analysis, and ML-assisted analysis will benefit drug development, early cancer detection and personalized treatment planning based on patient information.

Furthermore, this platform technology has thus a significant impact by allowing the detailed study and visualization of intravasation processes over time. It enables the tracking of cells and provides high throughput capabilities for drug development. Currently, over 90% of drugs fail in the clinical stage after years of costly development, primarily due to oversimplified in vitro models or non-translatable animal responses. With this technology, drug candidates and therapeutic approaches can “fail fast-fail early”, thereby reducing costs and the reliance on animal experiments. This capability not only benefits the efficiency of drug development but also aligns with ethical responsibilities.

The integration of ML with the inventors' EMT-driven lung cancer intravasation-on-a-chip model thus provides a physiologically relevant platform to mimic the initial process of cancer metastasis into microvasculature. This approach holds promise for improved drug development, personalized patient treatment plans and cancer progression prognosis.

The machine-learning based processing engine may be implemented as a processor within a computer server or the external computer which process the sampled data. In this embodiment, the system comprises a server which includes suitable components necessary to receive, store and execute appropriate computer instructions. The components may include a processing unit, including Central Processing Unit (CPUs), Math Co-Processing Unit (Math Processor), Graphic Processing Unit (GPUs) or Tensor processing united (TPUs) for tensor or multi-dimensional array calculations or manipulation operations, read-only memory (ROM), random access memory (RAM), and input/output devices such as disk drives, input devices such as an Ethernet port, a USB port, etc. Display such as a liquid crystal display, a light emitting display, or any other suitable display and communications links. The server may include instructions that may be included in ROM, RAM or disk drives and may be executed by the processing unit. There may be provided a plurality of communication links which may variously connect to one or more computing devices such as a server, personal computers, terminals, wireless or handheld computing devices, Internet of Things (IoT) devices, smart devices, edge computing devices, cloud devices. At least one of a plurality of communications links may be connected to an external computing network through a telephone line or other type of communications link.

The server may include storage devices such as a disk drive which may encompass solid state drives, hard disk drives, optical drives, magnetic tape drives or remote or cloud-based storage devices. The server may use a single disk drive or multiple disk drives, or a remote storage service. The server may also have a suitable operating system which resides on the disk drive or in the ROM of the server.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components, and data files assisting in the performance of specific functions, the skilled person will understand that the functionality of the software application may be distributed across a number of routines, objects, or components to achieve the same functionality desired herein.

It will also be appreciated that where the methods and systems of the present invention are either wholly implemented by computing systems or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. An apparatus for observation of biological activity in a sample, comprising a microfluidic device having a plurality of fluidic channels, including a center channel adjacent to a media channel defining a perfusable partition therebetween; wherein the center channel is arranged to facilitate mesenchymal transition (EMT) in biological cells housed within the center channel induced by a reagent supplied to the media channels.

2. The apparatus in accordance with claim 1, wherein biological cells in the center channel are undergoing epithelial to mesenchymal transition.

3. The apparatus in accordance with claim 2, wherein the center channel comprises a hollow luminal vasculature structure.

4. The apparatus in accordance with claim 3, wherein the hollow luminal vasculature structure is formed by vasculogenesis.

5. The apparatus in accordance with claim 4, wherein the hollow luminal vasculature structure is formed by culturing of human umbilical vein endothelial cells (HUVECs) to obtain a microvascular network of HUVECs.

6. The apparatus in accordance with claim 5, wherein the human umbilical vein endothelial cells and the biological cells housed within the center channel are co-cultured.

7. The apparatus in accordance with claim 6, wherein the human umbilical vein endothelial cells and the biological cells are co-cultured in a hydrogel composite injected into the center channel.

8. The apparatus in accordance with claim 1, wherein the reagent is an EMT-inducing cocktail composite.

9. The apparatus in accordance with claim 8, wherein the EMT-inducing cocktail composite includes TGFb1 and/or a macrophage condition medium.

10. The apparatus in accordance with claim 1, wherein each of the plurality of fluidic channel has an inlet port and an outlet port.

11. The apparatus in accordance with claim 1, wherein the microfluidic device comprises a plurality of partitioning structures arranged to separate the center channel from the media channel.

12. The apparatus in accordance with claim 11, wherein the partitioning structures includes triangular posts arranged in a regular interval.

13. The apparatus in accordance with claim 10, wherein the microfluidic device includes a pair of media channels sandwiching the center channel.

14. The apparatus in accordance with claim 13, wherein the pair of media channels are further connected with a common inlet port.

15. The apparatus in accordance with claim 6, wherein the biological cells include cancer cells selected from the group consisting of A549, NCI-H1975, and BEAS-2B cells.

16. The apparatus in accordance with claim 15, wherein the cancer cells are cultured as cancer spheroids.

17. The apparatus in accordance with claim 1, further comprising an imaging device adapted to capture images of the biological cells in the center channel to facilitate visualizing and recording intravasation events of the biological cells in the center channel.

18. The apparatus in accordance with claim 17, wherein the imaging device includes a microscopic imager.

19. The apparatus in accordance with claim 18, further comprising a machine-learning processing engine arranged to analyze the captured images to identify cancer intravasation events in the biological cells based on extracted features from the images.

20. A method for quantifying cancer intravasation events in a biological sample, comprising the steps of:
injecting and co-culturing HUVECs and cancer spheroids in the center channel of the apparatus in accordance with claim 7;
supplying an EMT-inducing cocktail composite in the media channel of the microfluidic device, the EMT-inducing cocktail composite including TGFb1 and/or a macrophage condition medium;
capturing microscopic images of the center channel at a predetermined time interval; and
processing the microscopic images using a machine-learning processing engine arranged to analyze the captured images to identify cancer intravasation events in the biological cells based on extracted features from the images to quantify an occurrence and characteristics of cancer intravasation events.
